(19) United States
(12) Patent Application Publication
(10) Pub. No.: US 2025/0110101 A1
(43) Pub. Date: Apr. 3, 2025

(54) PER AND POLYFLUOROALKYL SUBSTANCE MEASUREMENT

(71) Applicant: Dexsil Corporation, Hamden, CT (US)

(72) Inventors: Timothy Ryan LYNN, Hamden, CT (US); Theodore Brooks LYNN, Hamden, CT (US); Lawrence SACRAMONE, Waterbury, CT (US)

(73) Assignee: Dexsil Corporation, Hamden, CT (US)

(21) Appl. No.: 18/900,003

(22) Filed: Sep. 27, 2024

Related U.S. Application Data
(60) Provisional application No. 63/586,207, filed on Sep. 28, 2023.

(51) Int. Cl. G01N 33/24

(52) U.S. Cl. CPC G01N 33/24

(57) ABSTRACT

A method for detecting per and polyfluoroalkyl substances (PFAS) in a sample comprising preparing the sample in a manor for the specific matrix, taking the suitably prepared sample and subjecting it to a defluorination reaction, and measuring a fluoride concentration. A kit for detecting per and polyfluoroalkyl substances (PFAS) comprising a first container including a suitable extraction solvent, diluent, and/or extraction media, a second container including an isooctane butyl diglyme (BDG) mixture, a third container including 15% (w/w) naphthalene in BDG, a fourth container including non-ionic sodium metal, a fifth container including sodium acetate buffer, a filter pipette, and a test tube. Included can be a means for quantifying the fluoride.

### BACKGROUND

Due to its profligate use in manufactured goods and the unregulated disposal of waste, PFAS can be found throughout the environment even in some of the most remote waterways and soils of the world. Water has been identified as one of the most prevalent mass transfer methods for spread of PFAS and is also the most prevalent pollution vector to humans and ecosystems. Soils exposed to contaminated water or other wastes can retain PFAS contamination rendering them unfit for uses involving human contact.

Toxicity research has established there is no safe level of human exposure for essentially all PFAS compounds that have been thoroughly studied, leading to low parts per trillion (ppt) detection requirements. Evidence suggests that this toxicity may be conserved across the thousands of the lesser known PFAS compounds. Many of the lesser known PFAS compounds have established degradation pathways through weathering into toxic compounds and are called precursors as they determine the available source pollution.

The most sensitive and widely used lab methods are based on LC-MS/MS and only target specific PFAS compounds which can be isolated and for which standards are available. To fill the gaps between the small number of PFAS standards that are available for environmental labs and the 4000+ PFAS that are potentially contaminating the environment, it may be beneficial to be able to measure total fluorine by complete mineralization. Drinking and ground water methods aim for detection levels less than 100 ppt, however, many areas, such as heavy industrial and wastewater outflows, require measurement of total organic fluoride levels in the 50+ ppb range, and remediation levels for industrial sites may be closer to 500-600 ppb. Soil methods suffer from the same limitations with the added drawback that they all use an extraction step in order to get the PFAS into an organic solvent that can be used in the standard LC-MS/MS methods.

Environmental remediation projects often involve the mobilization of expensive machinery and manpower. In these situations, timely analysis of soil and water samples in the field or mobile lab can facilitate quicker turnaround and can dramatically lower costs. For this type of analysis, a total fluoride method can be used to track source removal efforts or monitor ground water or surface waters for the entire spectrum of PFAS contamination without the need for expensive lab equipment or long turnaround times for samples sent offsite. Total fluoride methods do not suffer from the specificity bias of the LC-MS/MS based methods.

One source of PFAS pollution is aqueous film forming foam (AFFF). AFFF enters the environment in multiple ways: through fire or catastrophic events, system discharge or false activation, firefighter training, and system testing. In areas where PFASs are not manufactured, PFAS groundwater contamination is typically traced back to a military fire or crash training sites and airports where AFFFs have been used. Perfluorooctane sulfonate (PFOS) is a long-chain PFAS found in older stocks of AFFF and as a breakdown product of precursor compounds. Perfluorooctanoic acid (PFOA) is also a long-chain PFAS. PFOA has been voluntarily phased out, but though not an intended ingredient in AFFF, is present as a side product created during the manufacturing process. Many AFFF formulations contain other unintended PFAS side products that have similar health and environmental concerns. Even with such concerns, an enduring challenge with AFFF is reliable and efficient detection of the broad range of PFAS present.

Due to a variety of reasons soil is a significant accumulation point for a large portion of the PFAS released into the environment. The exposure of soil to PFAS contamination can be due to the disposal of PFAS waste from any number of industrial processes, the land application of municipal sewage treatment sludge (biosolids) as fertilizer, and from the direct exposure to contaminated water through surface spills or leakage into ground water. Once exposed, soils will resiliently retain PFAS compounds, which make them difficult to analyze.

The trajectory of analytical methods for PFAS is determined almost entirely by regulation. Whether at the state, local or federal level, action levels, minimum concentration levels, clean-up levels, and reporting limits are determined by specific legislation or rule making. In many cases, such as with PFAS, the regulated limits can be compound specific and sometimes quite low. Therefore, when developing field analytical methods, it is important to demonstrate the ability to track the regulations. To achieve acceptable MDLs and a response profile that is of use to the regulated community, extraction methods and concentration steps must be optimized for each matrix so that field results can be correlated with lab results.

### SUMMARY

Wherefore, it is an object of the present invention to overcome the above-mentioned shortcomings and drawbacks associated with the current technology.

The system disclosed herein relates to products and methods for detecting PFAS in a sample by measuring the total organic fluorine content using a defluorination reaction and measuring a fluoride concentration. According to further embodiments, the defluorination reaction is a reductive defluorination reaction. According to further embodiments, the fluoride concentration is measured with an electrode. According to further embodiments, the electrode is a fluoride ion specific electrode. According to further embodiments, the PFAS concentration can be measured in concentrates containing PFAS by introducing the sample directly into the defluorination reaction, and measuring a fluoride concentration. According to further embodiments the method further comprises the step of extracting an aqueous sample with an immiscible solvent, drying the solvent and subjecting a portion of the solvent to the defluorination reaction. According to further embodiments comprising contacting a solid phase extraction (SPE) sorbent with the sample, subjecting the sorbent to a defluorination reaction, and measuring a fluoride concentration. According to further embodiments, extracting an aqueous sample by passing a known volume of the sample over an SPE, washing the SPE column and then eluting the PFAS from the column with a suitable solvent. The PFAS is then transferred to an aprotic solvent via solvent exchange or drying and resuspension and is then subjected to a defluorination reaction, and measuring the fluoride concentration. According to further embodiments, the method further comprises the step of conditioning the sorbent before mixing the sorbent with the sample. According to further embodiments, the sorbent is conditioned with a water miscible organic solvent. According to further embodiments, the sorbent is hydrophobic. According to further embodiments, the sorbent comprises either Granulated Activated Carbon (GAC) or polystyrene divinylbenzene. According to further embodiments, the sample is in an aqueous matrix. According to further embodiments, the method further comprises the step of equilibrating the sorbent after mixing the sorbent with the sample. According to further embodiments, the sorbent is contacted with the sample by vortexing for about 20 minutes creating a slurry. According to further embodiments, the method further comprises the step of drying the slurry to remove substantially all water from the slurry. According to further embodiments, the slurry is dried via a vacuum. According to further embodiments, the method further comprises the step of adding a solvent to the sorbent. According to further embodiments, the solvent is a mixture of an alkane and an ethylene oxide based glyme. According to further embodiments, the solvent is butyl diglyme (BDG), which can be mixed with isooctane up to a concentration of about 50% by weight to BDG. According to further embodiments, the sample is a soil matrix. According to further embodiments the sample is first contacted with an organic solvent mixture that can contain water, an alcohol, an ion paring reagent, NaCl, or a pH adjusting reagent. According to further embodiments the solvent is separated from the soil sample and dried over suitable drying agents such as NaCl and an aliquot is removed for analysis as above.

The presently disclosed invention further relates to methods and kits for detecting PFAS comprising a first container including a SPE sorbent and a second container including an organic solvent. According to further embodiments, the kit further comprises a third container including a mixture of naphthalene and a glycol diether. According to further embodiments, the kit further comprises a fourth container including elemental sodium, and a fifth container including buffer such as sodium acetate buffer, a filter pipette, and a test tube.

The presently disclosed invention also further relates to methods and kits for detecting PFAS comprising a first container including polystyrene divinylbenzene beads, a second container including 20% isooctane (w/w) in BDG, a third container including 15% naphthalene (w/w) in BDG, a fourth container including non-ionic sodium metal, a fifth container including sodium acetate buffer, a filter pipette, and a test tube.

The presently disclosed invention also further relates to methods and kits for detecting PFAS comprising a first container for collecting a water sample, an extraction column that contains either polystyrene divinylbenzene beads or granulated activated carbon, suitable wash solvents such as potassium nitrate and DI water, an elution solvent such as methanolic ammonium hydroxide, a second container to collect and dry the eluting solvent, a third tube including 20% isooctane (w/w) in BDG, a forth container including 15% naphthalene (w/w) in BDG, a fifth container including non-ionic sodium metal, a sixth container including sodium acetate buffer, a filter pipette, and a test tube.

In another system embodiment, a method of detecting PFAS in an aqueous sample may comprise contacting the aqueous sample, which may comprise groundwater, with a solid phase extraction media, which in varied embodiments may comprise granulated activated carbon (GAC) or macroporous polystyrene-divinylbenzene (PS-DVB) disposed within a column. The method may further comprise washing the solid phase extraction media with an aqueous solution while retaining most PFAS compounds on the solid phase extraction media. The aqueous solution may comprise potassium nitrate. The method may further comprise eluting any PFAS compounds from the solid phase extraction media with a polar solvent, which may be a non-aqueous polar solvent comprising an alcohol and a base. In some embodiments, the poler solvent may comprise methanolic ammonium hydroxide. The method may further comprise transferring any PFAS compounds from the polar solvent to a non-sodium reactive solvent. The method may further include chemically deflouringating any PFAS in the non-sodium reactive solvent, wherein defluorinating may include performing a reductive defluorination reaction, which may in turn comprise reacting any PFAS in the non-sodium reactive solvent with a reaction solvent, which may in turn comprise butyl diglyme (BDG) and a sodium dispersion. The sodium dispersion may comprise metallic sodium dispersed in oil. The method may further comprise transferring any fluoride ions which were defluorinated from the PFAS to an aqueous phase and measuring a concentration of fluoride in the aqueous phase. The measurement of concentration of fluoride ions in the aqueous phase may comprise measuring the fluoride concentration with a fluoride-ion specific electrode.

In some embodiments of the above method, the method may further comprise adding water to a sample vessel which contained or contains the aqueous sample, and transferring the water from the sample vessel to the column. In some embodiments, the water may be deionized water. In some embodiments, the method above may further comprise drying the non-polar solvent after eluting the solid phase extraction media. The drying may be done by blowing a stream of air, nitrogen, or other gas over the eluted sample.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIGS. 1A to 1F are structural diagrams of various PFAS compounds;

FIG. 2 is a table showing results of directly spiking concentrates to sodium reaction, to combustion mineralization, and compared to reported measurement by Houtz et al. for each PFAS source;

FIG. 3 is a graph showing recovery of AFFF spiked water by SPE method with coded analyte when compared to direct injection fluorine content over 0.5-10 ppm total fluorine range;

FIG. 4 is a plot and best fit line of the linear response of SPE method compared to direct concentrate injection covering full real sample spike database;

FIG. 5 is a graph showing recovery fraction of AFFF in water by cationic ion pairing method;

FIG. 6 is a plot and best fit lines of AFFF fluorine response by analyte for cationic ion paring method in water; and

FIG. 7 is a plot and best fit line of combined AFFF in water fluorine response by cationic ion pairing method. Outlier is sample code S11, a non-responder to the cationic pairing method.

FIG. 8 is a is a plot and best fit lines of PFAS fluorine response by analyte for anionic ion pairing method in water.

FIG. 9 is a graph showing extractable organic fluorine results using a GAC column.

FIG. 10 is a graph showing extractable organic fluorine results using a PS-DVB column.

FIG. 11 is a graph showing extractable organic fluorine results using a PS-DVB column with vacuum loading and methanol elution.

FIG. 12 is a graph showing the inorganic fluoride challenge of a GAC column.

FIG. 13 is a series of graphs showing sample recoveries for various PFAS analytes, distinguished by soil type.

FIG. 14 is a graph showing sample recoveries for various PFAS analytes, distinguished by soil type.

FIG. 15 is a graph showing theoretical and measured readings for individual analytes.

FIG. 16 is a graph showing the recovery rate of various PFAS analytes from the TOAB one-step soil-extraction method.

FIG. 17 is a series of graphs showing the recovery rate for various PFAS analytes in various soil types from the TOAB one-step soil-extraction method.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

The embodiments set forth below represent the necessary information to enable those skilled in the art to practice the invention and illustrate the best mode of practicing the invention. For the measurements listed, embodiments including measurements plus or minus the measurement times 5.0%, 10.0%, 20.0%, 50.0% and 75.0% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “substantially” means a value that is within +/−10.0% of the indicated value. In other embodiments, the value can be within +/−5.0% of the indicated value. In other embodiments, the value can be within +/−2.5% of the indicated value. In other embodiments, the value can be within +/−1.0% of the indicated value. In other embodiments, the value can be within +/−0.5% of the indicated value.

The presently disclosed invention also provides kits which are useful for carrying out the presently disclosed invention. The present kits comprise one or more containers containing the above-described assay components. The kit also comprises other containers containing solutions necessary or convenient for carrying out the invention. The containers can be made of glass, plastic or foil and can be a vial, bottle, pouch, tube, bag, etc. The kit may also contain written information, such as procedures for carrying out the present invention or analytical information, such as the amount of reagent contained in the first containers. The containers may be in another containers, e.g. a box or a bag, along with the written information.

In addition, the presently disclosed invention does not require that all the advantageous features and all the advantages of any of the embodiments need to be incorporated into every embodiment of the invention.

In one aspect, methods of detecting fluorinated compounds such as PFAS and PFOS are provided. The methods can include stripping fluorine atoms off of the organic backbones of the target analytes and measuring the resulting amount of fluoride ions that are realized. As the percent of fluorine in the target analytes is known or can be calculated, the total fluoride concentration can be used to determine the amount of fluorinated organic compounds reacted. As the target analyte may be a mixture of different compounds, the average number of fluorine atoms in the mixture of target compounds can be used to calculate the total amount of fluorinated organics. Analysis of the suspected source of the contamination can also be used to determine the fluorine percentage in the target analytes at a specific site.

Fluoride ion concentration can be measured in a number of ways including fluoride ion specific electrode (FISE), ion chromatography and colorimetric chemical tests. Fluoride ions can be removed from their organic backbones by, for example, alkali metal reaction and bomb calorimetry. Alkali metal reactions include reacting the sample with sodium metal or an organo sodium compound such as sodium naphthalenide, dioctyl sulfosuccinate sodium salt or sodium biphenyl. Components can be supplied combined or separately. For instance, sodium metal can be supplied as a dispersion in inert oil and the organic component, e.g., naphthalene, can be supplied dissolved in a solvent. The sodium can be supplied in a quantity sufficient to react with (consume) any water or other hydroxylated compounds that may be present and still be present in sufficient quantity to remove a substantial amount of the fluorine from the organic compounds. In various embodiments, the percentage of fluorine removed from the organic compound can be greater than 25%, greater than 50%, greater than 75% or greater than 90%. The amount of water in the solvent system reacting with the sodium can be less than 5%, less than 2%, less than 1% or less than 0.1% by weight.

The target analytes can be transferred to an appropriate solvent prior to reaction with an alkali metal compound. The solvent may be a solvent that does not react with the sodium compound. Such solvents are referred to herein as “non-sodium reactive solvents.” These solvents can include systems that are free of water, free of alcohols, free of aldehydes, free of esters and/or free of compounds with pendant hydroxyl groups. Examples include aprotic polar solvents, glycol diethers, aliphatic hydrocarbons, aromatic hydrocarbons and ethers. In some cases, the aprotic polar solvent can be mixed with an aliphatic or aromatic hydrocarbon. For example, the solvent can include a wt/wt ratio of aprotic polar solvent to hydrocarbon of greater than 9:1, greater than 8:2, greater than 1:1, greater than 1:2, less than 1:3, less than 1:2, less than 1:1 or less than 7:3, less than 8:2 or less than 9:1. In some cases, the aprotic solvent is a glycol diether and the hydrocarbon is a branched aliphatic hydrocarbon. The organic portion of the comingled solvent can include, for example, a glycol diether and a hydrocarbon. The hydrocarbon can be aliphatic or aromatic or cycloaliphatic and can be branched or linear. In some embodiments, the hydrocarbon includes n carbon atoms where n is from 4 to 20, 6 to 15, or 6 to 10. Specific hydrocarbons can include C₅, C₆, C₇, C₈, C₉ or C₁₀ branched or linear hydrocarbon The molecular weight of the hydrocarbon can be greater than 50, greater than 75, greater than 100, less than 500, less than 250 or less than 150.

In some embodiments a target compound is extracted from a soil sample using an aqueous solution. The aqueous solution is agitated with a soil sample to remove compounds of interest that have anionic or cationic constituents. In one set of embodiments an organic solvent system can be comingled with the aqueous solvent to provide a one step extraction to transfer PFAS compounds from soil to a non-sodium reactive solvent. Soil→aqueous phase→organic phase. The organic solvent system can be, for example, the aprotic polar solvent and hydrocarbon combination described above. The aqueous solution may contain at least one salt to aid in pairing with at least some of the PFAS compounds. For example, the aqueous solution can include an alkali metal salt, such as a monovalent alkali metal salt and/or a quaternary ammonium salt such as TBAHS or TOAB, a pH adjusting substance, or a miscible organic substance such as methanol. In some cases the quaternary ammonium salt includes organic R groups and can have a molecular weight of greater than 200, greater than 300, greater than 400 or greater than 500. The counterion for any salt is preferably not fluoride. The salt content can be, for example, greater than >1M, >5M, >10M, >20% by wt, >25% by wt or >40% by weight. The aqueous solution can be added in a quantity necessary to completely wet the soil being tested, and the soil can be saturated with water. With various soils, the ratio of water to soil can be greater than 1:10, greater than 2:10, greater than 3:10, greater than 4:10 or less than 1:1. The aqueous phase can also include a water miscible alcohol, such as methanol, ethanol or isopropanol. In some cases the alcohol content can comprise greater than 10%, 20%, 30%, 40% or 50% of the volume of the aqueous phase. In one set of embodiments the alcohol is MeOH.

In some aspects, soil and/or water samples are concentrated to improve the detection limit. For example, water samples can be contacted with an adsorbent, such as a solid phase extractant, to transfer and concentrate the target halogenated compounds. In some embodiments, the ratio of water to adsorbent can be, on a wt/wt basis, greater than 1:1, greater than 10:1, greater than 100:1 or greater than 1000:1. The adsorbent can be a material that retains targeted organic compounds when contacted with an aqueous solution include the target compounds. Adsorbents include, for example, materials that can selectively remove more than 50% of PFAS compounds from water. These include, for example, polystyrene divinyl benzene beads and granular activated carbon (GAC). Adsorbents can be mixed with the aqueous phase and filtered out or the aqueous phase can be passed through a column of adsorbent to remove target analyte.

In some cases, the adsorbent is reacted directly with an alkali metal compound and in some cases the target analytes are first eluted from the adsorbent prior to the reaction. The elution solvent can be a solvent that removes target analyte from the adsorbent but not necessarily inert to the alkali metal compound. Suitable elution solvents include, for example, basic alcohol solution. In some cases the reactive solvent can be completely removed via drying. In some cases this leaves the analytes on the inside of reaction tube as a film.

In some cases, after stripping fluorine off of the target compounds and converting it to fluoride the resulting aqueous and organic phases can be separated. This can be done for example, by centrifuge, settling and/or passing the aqueous phase through a filter, such as spun polypropylene/polyester or extruded cotton viscose to remove any residual organic phase from the aqueous phase.

In some embodiments, the reactants and components used in the extraction and reaction steps can be essentially free of fluorine and/or fluoride. For instance, the solvents, reactants and reaction vessels can contain less than 1 ppb, less than 100 ppt or less than 10 ppt, by weight of total fluorine. In some cases, fluoride from a source other than the target analytes can be removed from the system. For instance, fluoride that may be present in an aqueous solution that is passed through an adsorbent column can later cause interference in a test for target analyte. This fluoride can be removed from the system by passing a fluoride free aqueous solvent across the adsorbent and removing any fluoride that might be present. This can be done prior to elution of the organic target compounds and removes more than 50%, more than 75% or more than 90% of fluoride ions present and less than 25%, less than 10% or less than 5% of the target compounds from the adsorbent.

Some embodiments described herein utilize pH adjustment of the aqueous side of a liquid/liquid extraction of PFAS compounds. For example, a 0.46 M sodium bisulfate solution adjusted to approximately pH 0.3 with sulfuric acid can be used to adjust the pH of the aqueous extraction fluid. In other embodiments, this pH can be reduced to less than 3, less than 2 or less than 1.

Examples of PFSAs include the following compounds and their derivatives:

3H-Perfluoropentane-2,4-dione; 4,4-bis(Trifluoromethyl)-4-fluoropropanoic acid; Ammonium perfluorooctanoate; 3,5,6-Trichloroperfluorohexanoic acid; Perfluoro-3,7-dimethyloctanoic acid; 2-Perfluoropropyl-2-propanol; Allyl perfluoroisopropyl ether; Perfluorooctanamide; 2-(Perfluorohexyl)ethanol; 2-(Perfluorohexyl)ethylphosphonic acid; 1H,1H,5H-Perfluoropentyl methacrylate; Ethyl pentafluoropropionyl acetate; 2-Allyloxyperfluoroethanesulfonyl fluoride; 3H,3H-Perfluoropropyl triflate; 2,2,3,3,4,4,5,5,6,6,7,7,8,8,8-Pentadecafluorooctyl methacrylate; 1H,1H-Perfluorobutyl acrylate; 9:1 Fluorotelomer alcohol; Perfluorohept-1-ene; Perfluoroglutaryl difluoride; Perfluorooctanedioic acid; N,N-dimethyl-2H-perfluoroethanamine; 4:2 Fluorotelomer alcohol; Perfluorodiacetamide; 4-(1H,1H,2H,2H-Perfluorodecylthio) phenol; 1H,1H,6H,6H-Perfluoro-1,6-hexanediol; Perfluoro-1-octanesulfonyl chloride; 2-(Perfluorooctyl)ethyldimethylchlorosilane; Heptafluorobutyramide; 3-(Perfluoro-2-butyl)propane-1,2-diol; ((Perfluorooctyl)ethyl)phosphonic acid; Ethyl perfluorobutyl ether; Pentadecafluorooctanoyl chloride; (E)-Perfluoro (4-methyl-2-pentene); 1H,1H,5H,5H-Perfluoro-1,5-pentanediol diacrylate; 3-(Perfluoroisopropyl)-2-propenoic acid; 2,2,3,3,4,4,5,5,6,6,6-Undecafluorohexanal; Pentafluoropropanoic anhydride; 1,1,1,3,3-Pentafluorobutane; (1H,1H-Perfluoroethyl) (2H-perfluoroethyl)ether; Difluoromethyl 1H,1H-perfluoropropyl ether; Perfluoro-1,3,5-trimethylcyclohexane; 2H-Perfluoro-2-propanol; Perfluoro (4-methyl-3,6-dioxaoct-7-ene) sulfonyl fluoride; Bis(2,2,2-trifluoroethyl) sulfite; 1-Iodo-1H,1H,2H,2H-perfluoroheptane; (Perfluoroethyl)methyl iodide; 1-Iodopentadecafluoroheptane; 2,2,3,3-Tetrafluoropropyl trifluoroacetate; 4-(Perfluorobutyl)-2-butanone; 1H,1H-Perfluoro-3,6,9-trioxadecan-1-ol; 2H,2H,3H,3H-Perfluorooctanoic acid; Perfluorooctanesulfonic acid; Potassium perfluorobutanesulfonate; Sodium perfluorooctanoate; 1,4-Dibromo-1,1,2,2-tetrafluorobutane; Heptafluoropropyl iodide; Perfluoro-3,6,9-trioxaundecane-1,11-dioic acid; Methyl perfluorohexanoate; Methyl perfluoro (3-(1-ethenyloxypropan-2-yloxy)propanoate); 2-Iodoperfluorobutane; 3H,3H-Perfluoro-2-butanone; Ethyl perfluorobutanoate; 2,2,2-Trifluoroethyl triflate; 4,4,5,5,6,6,7,7,8,8,9,9,9-Tridecafluorononanoic acid; 1H,1H-Heptafluorobutyl triflate; 1H,1H,3H-Perfluoropropyl triflate; Perfluorodecanoic acid; Tris(2,2,2-trifluoroethyl) borate; 6:1 Fluorotelomer alcohol; Perfluoro-2,3-dimethylbutane; Perfluoroundecanoic acid; 2:1 Fluorotelomer alcohol; Perfluorooctanoic acid; Methyl perfluoro-3,6-dioxaheptanoate; Hexafluoroamylene glycol; Perfluorobutyraldehyde; 1H,1H-Perfluoropropyl methacrylate; 2-(4H-Perfluorobutyl)-2-propanol; Flurothyl; Perfluoro-3,6-dioxaoctane-1,8-dioic acid; 1H,1H,5H-Perfluoropentyl acrylate; 2H-Perfluoro-5-methyl-3,6-dioxanonane; Perfluoro (2-bromoethoxy)ethene; 4H,4H-Perfluoro-6,6-dimethylheptane-3,5-dione; 1-H-Perfluorodecane; Perfluorononanoic acid; (Perfluorobutyryl)-2-thenoylmethane; 3-(Perfluorobutyl)-1-propanol; 1-Iodo-1H,1H,2H,2H-perfluorononane; 1,1-bis(Trifluoromethyl)methoxy-2-ethanol; Perfluorodecyl iodide; 1H,1H,9H-perfluorononyl methacrylate; Methyl perfluorobutanoate; 3-(Perfluoro-2-butyl)propanoic acid; Sodium perfluorodecanesulfonate; (Perfluorooctyl)propyl acetate; 1H,1H-Perfluoropentylamine; Methyl 3,3,3-trifluoro-2-(trifluoromethyl)propionate; Bis(1H, 1H-perfluoropropyl)amine; Dichloromethyl((perfluorohexyl)ethyl)silane; Perfluoromethylcyclopentane; 4:4 Fluorotelomer alcohol; Perfluorobutanesulfonic acid; Perfluoropentanedioic acid; 2,2,3,3,4,4,4-Heptafluorobutyl methacrylate; 6-(Perfluorohexyl) hexanol; 1H,1H-Perfluorohexylamine; Perfluorononanoyl chloride; Perfluoro-1-decene; Perfluorooctanoyl fluoride; 2,2,2-Trifluoroethyl perfluorobutanesulfonate; 1H,2H-Hexafluorocyclopentene; Perfluoro-tert-butyl isobutyrate; 1H,6H-Perfluorohexane; Perfluorohexanesulfonic acid; Perflubrodec; Perfluoro-1-iodohexane; Perfluorobutanol; (Perfluorocyclohexyl)methanol; Perfluoro (2-(2-propoxypropoxy)-1H, 1H-propan-1-ol); N-Methyl-N-trimethylsilylheptafluorobutyramide; 2H-Perfluoro (2-methylpentane); Hexafluoroisopropyl methyl ether; 1-(Perfluorohexyl) octane; 1-Bromopentadecafluoroheptane; Perfluorooct-1-ene; Perfluorohexanesulfonamide; Perfluoromethyldecalin; Perfluamine; 1H,1H,5H-Perfluoropentyl-1,1,2,2-tetrafluoroethylether; Perfluoro-2,5-dimethyl-3,6-dioxanonanoic acid; Triethoxy ((perfluorohexyl)ethyl)silane; 3,3-Bis(trifluoromethyl)-3-hydroxypropionic acid; N-Methyl-N-(2-hydroxyethyl) perfluorooctanesulfonamide; Methyl 2H,2H,3H,3H-perfluoroheptanoate; 2-Vinylperfluorobutane; Perfluoro-1,4-diiodobutane; 1,4-Divinylperfluorobutane; 2-(Perfluoro-3-methylbutyl)ethyl methacrylate; 3:1 Fluorotelomer alcohol; N-(Phenylmethyl) perfluorobutanamide; 1H,1H,7H-Perfluoroheptyl 4-methylbenzenesulfonate; Methyl heptafluoropropylketone; Perfluoropropanoic acid; (1S,4S)-3-(Heptafluorobutyryl) camphor; Perfluorobutanoic anhydride; Ethyl perfluorooctanoate; Methyl 1H,1H-perfluoropropyl ether; Difluoromethyl 2H-perfluoropropyl ether; 1H,1H,3H-Perfluorobutyl 2-methylacrylate; (2H-Perfluoroethyl) (1H,1H,3H-perfluoropropyl)ether; 6:2 Fluorotelomer phosphate monoester; Perfluoro-3,6-dioxadecanoic acid; Perfluoro-1-butanesulfonyl chloride; 3H,3H-Perfluoroheptane-2,4-dione; Potassium perfluorohexanesulfonate; 1H,1H,10H,10H-Perfluorodecane-1,10-diol; 3-(Perfluoropropyl)propanol; Perfluorobutanesulfonyl fluoride; 2:2 Fluorotelomer alcohol; Ethyl perfluoropentanyl ketone; 5-Iodoperfluoro-3-oxapentanesulfonyl fluoride; 5-Bromo-4,4,5,5-tetrafluoropentanoic acid; Perfluoroheptanes (branched and linear); 2,4,6-Tris(pentafluoroethyl)-1,3,5-triazine; 2,3-Dichlorooctafluorobutane; Perfluorobutyrylamidine; 7:3 Fluorotelomer alcohol; (Perfluorooctyl)propanoyl chloride; Perfluoroadipoyl chloride; 5H-Octafluoropentanoyl fluoride; Perfluoro-2-butyltetrahydrofuran; Perfluoroheptanoic acid; 1,6-Divinylperfluorohexane; 2-Bromo-2-(Perfluorohexyl)ethene; 1,6-Dibromododecafluorohexane; Sevoflurane; 3:3 Fluorotelomer carboxylic acid; Perfluorotridecanoic acid; 2-Iodo-1h,1h,2h,3h,3h-perfluorodecan-1-ol; Perfluoro-3,6,9-trioxadecanoic acid; 1H,1H-Perfluorobutyl perfluorobutanesulfonate; 1-(Perfluorofluorooctyl)propane-2,3-diol; 1,8-Diiodoperfluorooctane; 2-(Trifluoromethoxy)ethyl trifluoromethanesulfonate; 2 (Perfluoro-2-propyl)ethanol; Ethyl 2H-perfluoropropyl ether; 1H,1H,6H,6H-Perfluorohexane-1,6-diol diacrylate; Perfluoropentanoic acid; 2-(2-Iodoethyl) perfluoropropane; 3-Methoxyperfluoro (2-methylpentane); Perfluoro-3,6-dioxaheptanoic acid; Perfluorohexylchloride; (2H-Perfluoroethoxy)methyloxirane; 3-(Perfluoro-1-propyl)-1,2-propanediol; 2-Aminohexafluoropropan-2-ol; 1H,1H,11H,11H-Perfluorotetraethylene glycol; Perfluorooctanesulfonate; 1H,1H-Perfluoro-1-pentanol; 6:2 Fluorotelomer sulfonic acid; Perfluoropentanamide; Perfluorooctanamidine; 2,2,3,3-Tetrafluoropropyl ether; Perfluorocyclohexanecarbonyl fluoride; 3-(Perfluoroethyl)propanol; 1H,1H,9H-Hexadecafluoro-1-nonanol; (Perfluorohexyl)methyl methacrylate; Perfluorohexanoic acid; N-(3-(Dimethylamino)propyl) perfluorohexane sulfonamide; Trifluoroacetyl triflate; 2-(Perfluorooctyl)ethyl methacrylate; Ethyl perfluorononanoate; Nonafluoro-tert-butanol; 8H-Perfluorooctanoic acid; 3-(Perfluorobutyl)propanoic acid; (Heptafluorobutanoyl) pivaloylmethane; 3:2 Fluorotelomer iodide; 1H,1H-Heptafluorobutylamine; Methyl perfluorooctanoate; 3,3,4,4,5,5,6,6,6-Nonafluorohexene; Methyl perfluoro (2-propoxypropanoate); 7:1 Fluorotelomer alcohol; Hexaflumuron; 2-(Perfluorobutyl)ethyl acrylate; Perfluorodecanedioic acid; Perfluoro-2-ethoxyethanesulfonic acid; (Perfluoro-n-octyl)ethane; 3-(Perfluorohexyl)propanol; Perfluorobutanedioic acid; Perfluoro (2-methyl-3-oxahexanoyl) fluoride; 1-(Perfluorohexyl)ethane; 2,2,2-Trifluoroethyl trifluoroacetate; 3,3-Bis(trifluoromethyl)-2-propenoic acid; Methyl perfluoroethyl ketone; Perfluoroheptanoyl chloride; Perfluoro-4-isopropoxybutanoic acid; Hexafluoroisopropyl acrylate; Perfluorooctanesulfonamide; Methyl 3-chloroperfluoropropanoate; Bis(2,2,2-trifluoroethyl)amine; Dimethoxymethyl((perfluorohexyl)ethyl)silane; 1H,1H,3H-Perfluorobutanol; 3-(Perfluorooctyl)propanol; [(Heptafluoropropyl)sulfanyl]acetic acid; 2:2 Fluorotelomer iodide; 2-(Perfluoropropoxy)-1H, 1H-perfluoropropanol; Perfluoro-3,6,9-trioxatridecanoic acid; 1,1,2-Trifluoro-1-methoxy-2-(trifluoromethoxy)ethane; Methyl perfluoropentanoate; 2-Chloro(perfluoro-2-methylpentane); 3-Ethoxyperfluoro(2-methylhexane); Trifluoromethanesulfonic acid; 1H,2H,2H-Perfluorobutane; Hexafluoroacetylacetone dihydrate; 3,5,7,8-Tetrachloroperfluorooctanoic acid; 3(Perfluoro-2-butyl)propanol; 9-Chloro-perfluorononanoic acid; 3-(Perfluorooctyl)-1,2-propenoxide; N,N-Diethyl-2H-perfluoropropanamine; Methyl 4H-perfluorobutanoate; 1H,1H,11H-Eicosafluoro-1-undecanol; Perfluoro(N-methylmorpholine); Hexafluoropropene oxide trimer; Perfluoro-2-methyl-3-pentanone; Perfluoro(4-methoxybutanoic acid); 1H,1H,9H-Perfluorononyl acrylate; 2,2,2-Trifluoro-N-(2,2,2-trifluoroethyl) acetamide; Decafluorocyclohexene; Methyl 2H,2H-perfluorobutyl ether; Diethyl perfluoroglutarate; Perfluoro-1,2-dimethylcyclohexane; 2H-Perfluoroisobutyric acid; Perfluoro-3-(1H—N-Ethylperfluorooctanesulfonamide; 1,6-perfluoroethoxy)propane; Diiodoperfluorohexane; 1H-Perfluorohexane; 1H,1H,2H,2H-Perfluorohexyl methacrylate; (Heptafluoropropyl trimethylsilane; 11:1 Fluorotelomer alcohol; Nonafluoro-1-iodobutane; 4-[3-(Perfluorobutyl)-1-propyloxy]benzyl alcohol; 5H,5H-Perfluoro-4,6-nonanedione; Trifluoroacetic acid; 2,2-Difluoroethyl triflate; Cyclohexafluoropropane-1,3-bis(sulfonyl)imide; 4H-Perfluorobutanoic acid; 1-Propenylperfluoropropane; 1H,1H,1H,2H-Perfluoro-2-heptanol; 3-(Perfluoro-3-methylbutyl)-1,2-propenoxide; Trifluoromethyl trifluoromethanesulfonate; 1H,1H,7H-Dodecafluoro-1-heptanol; 3H-Perfluoro-4-hydroxy-3-penten-2-one; 1H,1H,5H-Perfluoropentanol; 1-Chloro-6-iodoperfluorohexane; Bis(perfluoroisopropyl)ketone; 2-Vinyl(1-bromoperfluoroethane); Methyl perfluoropentyl ketone; Octafluoroadipamide; 2-(Perfluorobutyl)ethanethiol; Methyl tetrafluoro-2-(trifluoromethyl)propionate; (6H-Perfluorohexyl)methyl acrylate; 3-(Perfluoroheptyl)propanoic 3-(Perfluoroisopropyl)-(2E)-difluoropropenoic acid; Perfluoromethylcyclohexane; Perfluoropinacol; ((2,2,3,3-Tetrafluoropropoxy)methyl)oxirane; 1H-Perfluoro-1,1-propanediol; 1,2-Dibromohexafluoropropane; 2,2-Bis(trifluoromethyl)propionyl fluoride; Perfluorohex-1-ene; Perfluorobutanesulfonic anhydride; Methyl 3H-perfluoroisopropyl ether; 2H-Hexafluoropropyl allyl ether; Perfluoroisohexane; 1H, 1H,2H-Perfluoro-1-decene; 1,2-bis(1,1,2,2-Tetrafluoroethoxy)ethane; 1H,1H-Perfluorooctylamine; 2-(Perfluorooctyl)ethyl dihydrogen phosphate; Ethyl perfluoroheptanoate; Perfluoro-(2,5,8-trimethyl-3,6,9-trioxadodecanoic) acid; 2-(Perfluorohexyl)ethyl methacrylate; Perfluoro-3,6-dimethyl-1,4-dioxan-2-one; 1-Bromoperfluorobutane; Perfluorononanedioic acid; 1-Bromoheptafluoropropane; 2-(Perfluorohexyl)ethanethiol; 3H,3H-Perfluoro-2,4-hexanedione; Trichloro((perfluorohexyl)ethyl)silane; Perfluoroisobutyl methyl ether; 3:2 Fluorotelomer alcohol; 1H,1H-Perfluorooctyl acrylate; 1-Pentafluoroethylethanol; 1H,1H,8H,8H-Perfluorooctane-1,8-diol; Ethyl perfluoropropionate; 3-(Perfluoroisopropyl)propanol; 1-Hydroperfluoroheptane; Perfluoroheptanesulfonic acid; Difluoromethyl 2,2,3,3-tetrafluoropropyl ether; Hexafluoroglutaryl chloride; 2H-Perfluoroisopropyl 2-fluoroacrylate; N-Methylperfluorooctanesulfonamide; 8:1 Fluorotelomer alcohol; 1H, 1H,2′H-Perfluorodipropyl ether; tris(Trifluoroethoxy)methane; Perfluorodecane; N,O-Bis(trifluoroacetyl)hydroxylamine; 7H-Perfluoroheptanoyl chloride; Ethyl 5H-octafluoropentanoate; Heptafluorobutyl iodide; Ethyl perfluoropentanoate; Perfluorobutyl methyl ether; 9H-Perfluorononanoic acid; Methyl pentafluoropropionate; (6H-Perfluorohexyl)methyl methacrylate; Perfluorobutanoic acid; (2H-Perfluoropropyl)(1H,1H-perfluoroethyl)ether; Perfluoro-1,3-dimethylcyclohexane; Perfluorooctane; 1H,1H-Perfluoropropylamine; Perfluorotetradecanoic acid; N-Ethyl-N-(2-hydroxyethyl)perfluorooctane sulfonamide; 2-(Perfluorooctyl)ethyl acrylate; Methyl 4H-perfluorobutyl ketone; 1H, 1H-Perfluoropropyl acrylate; 2-(Perfluorobutyl)-1-ethanesulfonic acid; Hexafluoro-2-methyl-2-propanol; Pentafluoropropylamidine; 1-Iodoperfluoropentane; Perfluorodimethylcyclobutane; Trimethoxy((perfluorohexyl)ethyl)silane; Potassium perfluorooctanoate; Potassium perfluorooctanesulfonate; Perfluorohexylbromide; 2,3,3,3-Tetrafluoro-2-(perfluoropentoxy)propan-1-ol; N-[(Perfluorooctylsulfonamido)propyl]-N,N,N-trimethylammonium iodide; Perfluoropropyl trifluorovinyl ether; Pentafluoroallyl fluorosulfate; 2(2H-Perfluoro-2-propyl) acetic acid; 1,8-Divinylperfluorooctane; 1H,1H,2H-Perfluorocyclopentane; 5H-Perfluoropentanal; Perfluoro-3-methoxypropanoic acid; Heptafluorobutyryl Chloride; 1-(Perfluorohexyl)-2-iodopropane; 6H-Perfluorohex-1-ene; Trimethylsilyl perfluorobutanesulfonate; (Perfluoro-5-methylhexyl)ethyl 2-methylprop-2-enoate; Perfluorooctanesulfonyl fluoride; 1H,1H-Perfluorononylamine; 1-Perfluoropropylethanol; 1H,1H-Heptafluorobutyl epoxide; 7H-Perfluoroheptanoic acid; (Perfluoro-3-methylbutyl)-2-hydroxypropyl acrylate; 3H-Perfluorobutanoic acid; 2-(Perfluorooctyl)ethanthiol; 4-((Perfluorohexyl)ethyl)phenylmethanol; 8:3 Fluorotelomer carboxylic acid; Nonafluoropentanamide; 1,1,2,2-Tetrafluoro-3-iodopropane; 2-Amino-2H-perfluoropropane; Heptafluoro-2-iodopropane; Trifluoroacetic anhydride; Perfluorohexanedioic acid; 3-(2,2,3,3-Tetrafluoropropoxy)prop-1-ene; 1H,1H,8H-Perfluoro-1-octanol; 3H-Perfluoro-2,2,4,4-tetrahydroxypentane; N-Methylperfluoroheptanamide; Perfluorooctanedioic diamide; Perfluorosuccinic anhydride; 2H,3H-Decafluoropentane; 2,2,3,3-Tetrafluoropropyl acrylate; 11-H-Perfluoroundecanoic acid; (1R,4R)-3-(Heptafluorobutyryl)-camphor; Perfluoro-2-methyl-3-oxahexanoic acid; 2-(Perfluorooctyl)ethanol; N-Methyl-bis-heptafluorobutyramide; 2,2-bis(Trifluoromethyl)-2-hydroxyacetic acid; Perfluoro-1,2-dimethylcyclobutane; Methyl 5H-perfluoropentanoate; Pentafluoropropionamide; 1H,1H-Perfluoroheptylamine; 1H,8H-Perfluorooctane; 5:1 Fluorotelomer alcohol; (Perfluorocyclohexyl)methyl prop-2-enoate; 8:2 Fluorotelomer sulfonic acid; 2H-Perfluoropropanoic anhydride; 1H,1H,8H,8H-Perfluoro-3,6-dioxaoctane-1,8-diol; 3-(Perfluorohexyl)-1,2-epoxypropane; and (Perfluoroheptyl)ethene; Perfluoro-15-crown-5-ether; Methylperfluoro-2,5,8-trimethyl-3,6,9-trioxadodecanoic acid; trans-1,2-Bis(perfluorohexyl)ethylene; 2-(Perfluorodecyl)ethanol; Perfluorotridecane; Perfluorohexadecanoic acid; 1-Bromoperfluorononane; (Perfluorohexyl)ethyl acrylate; 1H,1H,2H,2H-Perfluorooctyl iodide; Methyl perfluorobutyl ketone; 3-(Perfluorooctyl)propyl iodide; ((Perfluorodecyl)methyl) oxirane; Ammonium perfluoro-2-methyl-3-oxahexanoate; 11-(Perfluoro-n-octyl) undec-10-en-1-ol; ((Perfluorooctyl)ethyl)di(propan-2-yl)silane; 2H-Perfluoro (5,8-dimethyl-3,6,9-trioxadodecane); Perfluorododecane; Bis(3,3,4,4,5,5,6,6,7,7,8,8,8-tridecafluorooctyl) hydrogen phosphate; Perfluoro (1,3-dipropoxycyclohexane); Perfluorooctadecanoic acid; 5H-Octafluoropentanoic acid; (Perfluorooctyl)ethyltrichlorosilane; 10:1 Fluorotelomer alcohol; 1-Chloro-8-iodoperfluorooctane; 1H,1H,2H,2H-Perfluorodecyltrimethoxysilane; 1H,1H,2H,2H-Perfluorododecyltrichlorosilane; Perfluorododecanoic acid; Perfluorooctane sulfonamido amine; 2-(Perfluorodecyl)ethyl acrylate; 1H,1H,2H,2H-Perfluorodecyltriethoxysilane; 10:2 Fluorotelomer methacrylate; Bis(2-(perfluorooctyl)ethyl) hydrogen phosphate; 2,4,6-Tris(heptafluoropropyl)-1,3,5-triazine; 1-(Perfluorooctyl)-2-iodoethane; 10:2 Fluorotelomer iodide; 1-(Perfluoroheptyl)-2-iodopropane; (Perfluorododecyl)ethylene; (Perfluorodecyl)ethylene; Perfluorotributylamine; Perfluoro-1-ethyl-3-propoxycyclohexane; ((Perfluoro-9-methyldecyl)methyl)oxirane; Perfluoro-2,5,8-trimethyl-3,6,9-trioxadodecanoyl fluoride; Perfluorooctyl iodide.

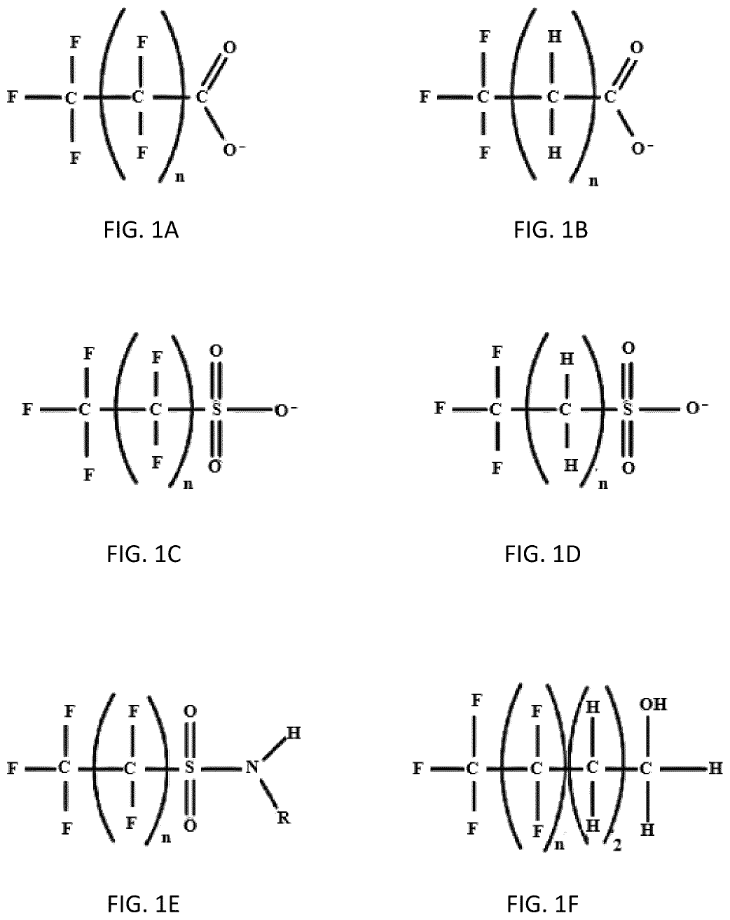

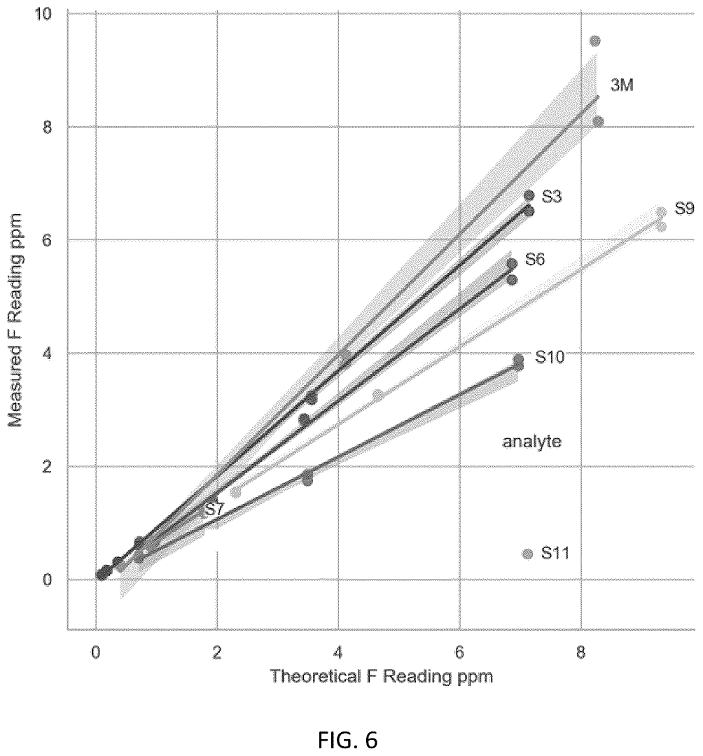

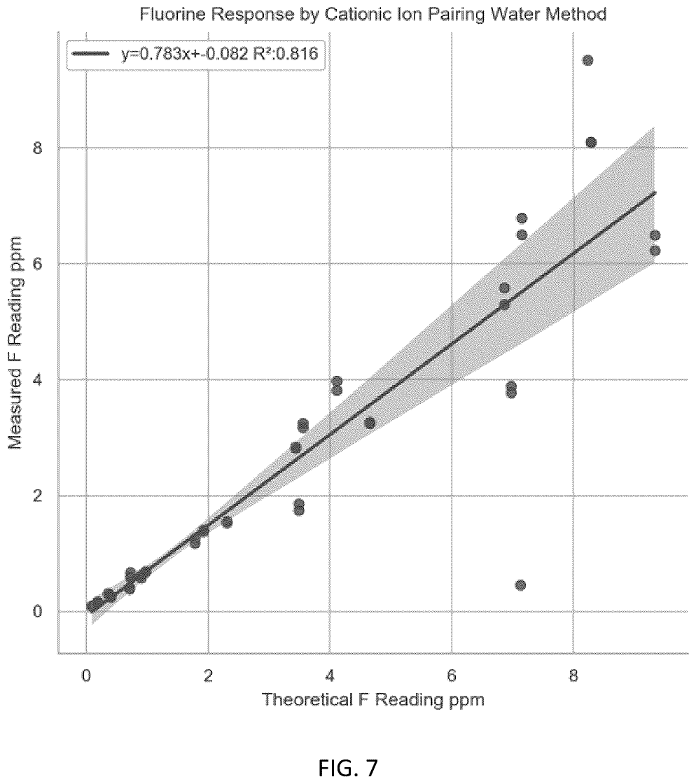

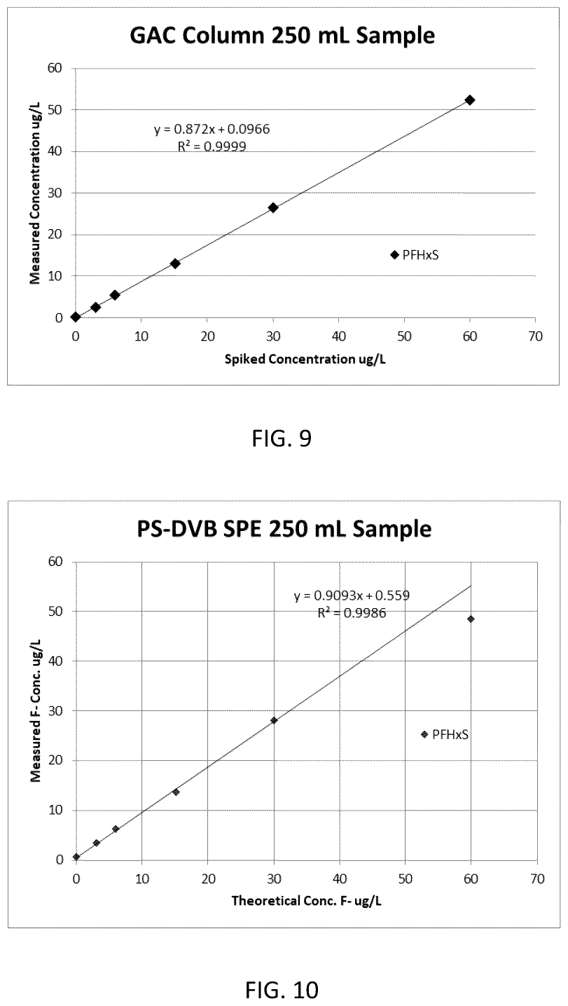

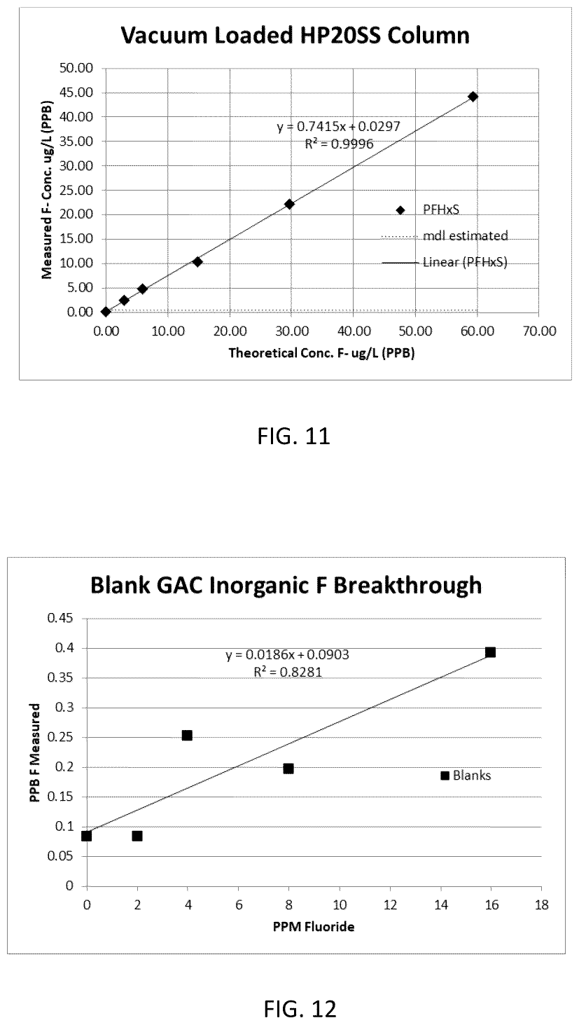

Turning now to FIGS. 1-17, a brief description concerning the various components will now be discussed.

To evaluate the ability of the disclosed methods to analyze for AFFF, AFFF water mixtures were tested after being collected from an actual AFFF foam system cleanup and retrofit that required a minimum detection limit of 0.5 ppm in the lines before a new system could be charged. These AFFF systems are used throughout hangers, refineries, and many DoD sites.

With the samples of AFFF water mixtures, reductive defluorination coupled with fluoride ion specific electrode (FISE) detection was used to quantify the AFFF. The disclosed method uses reactive liberation of fluoride and quantification with a fluoride ion specific electrode FISE after preparing test samples from aqueous matrices. The approach led to the investigation of several classes of PFAS that possess differing extraction tendencies from aqueous matrices. Ion pairing serves as a powerful extraction aide for analytes based on compatibilities with headgroup charge, either cationic or anionic. However, many AFFF foams were found to have organic fluorine sources that adsorbed universally to SPE materials. It is believed that this is because AFFF technologies include many different classes of fluorinated material, for example anionic, cationic, zwitterionic, non-ionic, and polymeric. Due to this behavior, a method was developed using solid phase extraction (SPE) in order to target all of the classes of PFAS based on surface activity. It was found that many of the AFFF foam concentrates have fluorine sources that are essentially irreversibly retained on the SPE sorbent, meaning that once sorbed from an aqueous sample they could not be efficiently extracted into a liquid phase for subsequent analysis. Instead of relying on liquid extraction from the SPE, the entire SPE sorbent phase (a solid) was subjected to reductive defluorination. Once SPE is employed directly, the detection limit is only limited by the size of the water sample that can be extracted with the SPE.

### Experiments:

Alkali metal reaction and quantification (Procedure):

The sodium based defluorination/quantification experiments referred to herein use the following procedure unless otherwise noted.

1.0 mL 15% (w/w) naphthalene in butyl diglyme (BDG) catalyst is added to a reaction tube and shaken manually for 10 seconds. A glass sodium ampule containing 0.4 g of a 20% Na metal suspension in light oil is added to the reaction tube, crushed and reacted with the mixture for 1.0 minute while shaking intermittently. The reaction is quenched with 7 mL of an aqueous sodium acetate buffer solution and shaken for 2 minutes to extract fluoride. The reaction tube is vented and turned upside down on its cap, allowing the phases to separate for 2 minutes. The aqueous extract is passed through a fibrous, fluoride free filter into a fresh test tube and read with a calibrated FISE connected to an L2000 halogen detection system (Dexsil).

### Example 1: AFFF Concentrate Direct Spikes

Samples of AFFF foam were obtained from either marked surplus concentrate containers or remediation activities on charged fire suppression systems. These concentrates did not easily dissolve in a butyl diglyme based reaction solvent. To obtain a total fluorine concentration on the samples, the AFFF samples were spiked directly to sodium reaction tubes at 20-40 mg. (This represents about a 1:2 molar ratio of water:sodium so that some sodium survives the reaction with water.) These spike levels still maintained the reactivity of the system and did not overwhelm the reaction with protic material. By reacting the pellet of AFFF concentrate for 10 minutes with 40 mg of dioctyl sulfosuccinate sodium salt (AOT) the material was fully reacted, meaning the organically bound fluorine atoms had been stripped from the organic backbones and converted to fluoride ions. After the reaction, the quenching and extraction of free fluoride continued as described previously. Readings were made on the FISE calibrated at 1 ppm fluoride.

To validate the defluorination efficiency of precursor material by the sodium naphthalenide reaction, the samples were also mineralized through combustion and quantified with the FISE. The combustion method was completed as follows. 0.1 g of sample was weighted into a crucible along with 0.5 g of methyl tert butylether and up to 1.0 g total with butyldiglyme to aid in combustion. The bomb was charged with 29 atm of oxygen and ignited with a nickel fuse. Fluoride was then extracted with Tisab buffer and measured with FISE.

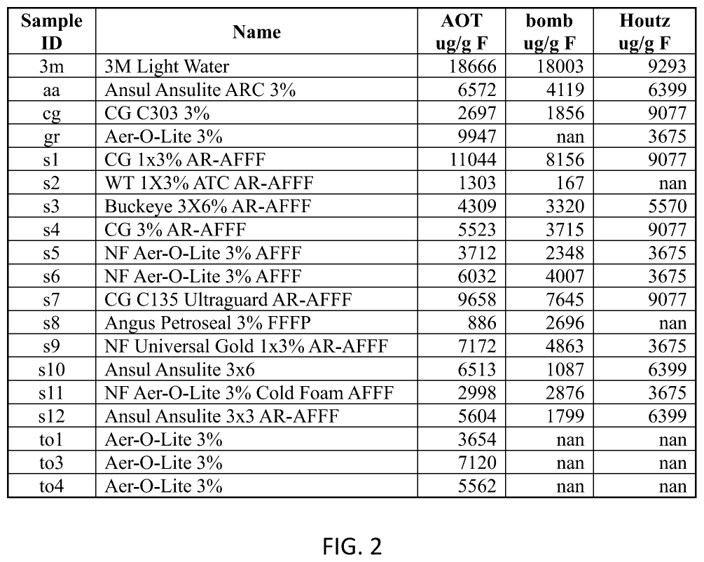

Results: The results of directly spiking AFFF concentrate material into reaction, bomb combustion method, and extracted total fluoride values from Houtz et al. research are summarized in FIG. 2, which also includes sample ID's.

Directly spiking AFFF concentrates to measure the total organic fluorine load is difficult because of the competing problems of 1) incomplete dissolution and maintaining reactivity in BDG, and 2) the need to avoid all extraction procedures in order to eliminate any possible losses for the AFFF samples. The protocol was changed to lengthen the reaction time and to add an oil soluble surfactant, such as sodium docusate (AOT) additive, for AFFF concentrates due to their tendency to form a non-reactive micelle in the BDG solvent. It is believed that the AOT, surprisingly, acts as a surfactant aiding in breaking down the micelle structure in the reaction environment. The AOT amended reaction recovery agreed with the bomb combustion measurements. One obstacle with AFFF extraction is the co extraction of water up to the saturation level of water in BDG, which is around 1.5% wt. This overloaded the subsequent drying steps to prepare for the moisture sensitive defluorination reaction. This was overcome by the addition of 20% wt isooctane to in BDG. The measurements are also the same magnitude as Houtz et al. measured in their extensive analysis of precursor levels in aged commercially available concentrate samples. The weathering and unique history of the concentrate samples obtained was unknown, therefore, the magnitude of results was used to validate the procedure. The measured fluorine levels at the optimized reaction condition were then used as the comparators for all reported recoveries of AFFF extraction methods.

### Example 2: Liquid/Liquid Extraction: Cationic Ion Pairing Method for Water

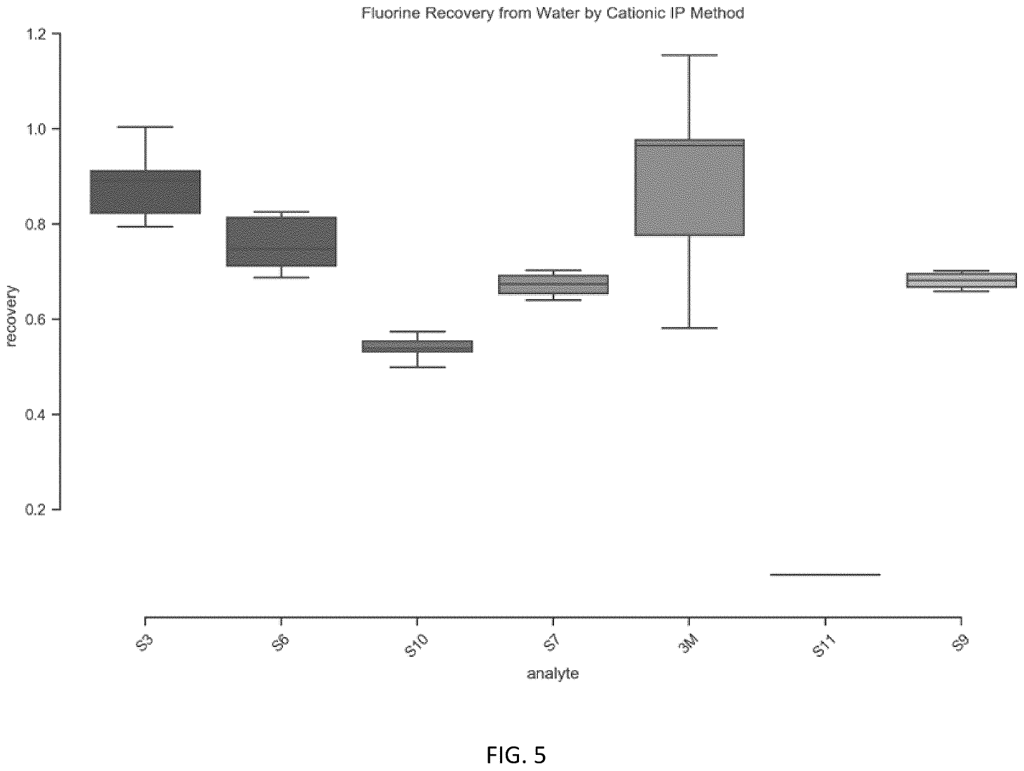

Cationic head groups are commonly found in foam concentrates due to their excellent foaming performance. These head groups should be paired with an anionic ion to facilitate a paired extraction. To a 50 mL centrifuge tube containing 1.0 gram of NaCl, 10 mL of AFFF standard was added along with 0.93 mL 3.0 M phosphoric acid and 0.07 mL 3.0 M NaH₂PO₄, 100 μL 40 mg/mL AOT in 20% (w/w) isooctane in butyl diglyme. The mixture was shaken for 5 minutes and allowed to settle. An organic layer was removed and slurried over NaCl to dry. Five mL of the dry extract was transferred to a reaction tube, reacted and quantified. Results are provided in FIGS. 5-7 which indicate excellent correlation between the cationic ion pairing method and the theoretical values.

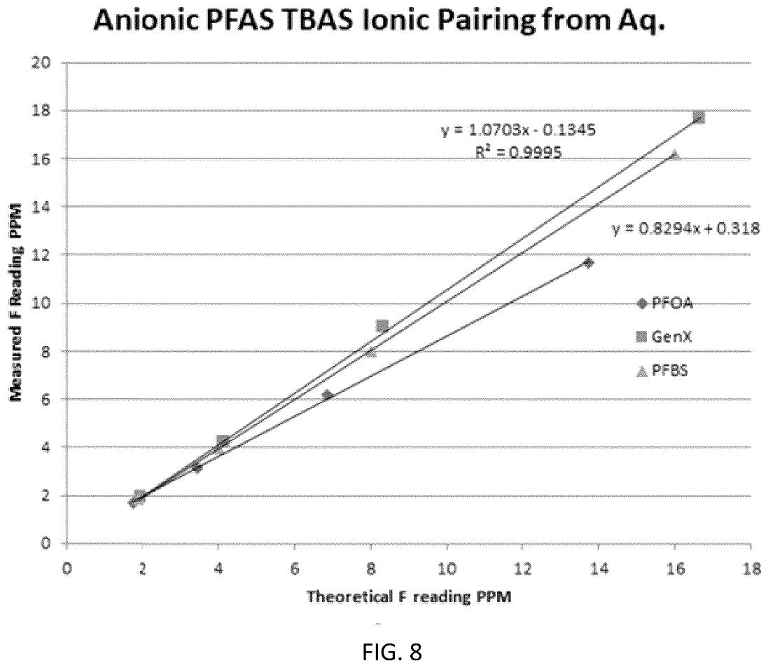

Liquid/Liquid Extraction: Anionic ion pairing for water: Anionic head groups are also common in foam concentrates. The recovery of anionic PFAS from water can be achieved using a cationic ion pairing agent such as tetra butylammonium hydrogen sulfate (TBAHS) with BDG based solvent. Aqueous samples are spiked with anionic PFAS and to this sample mixture is added 3.0 mL ionic pairing solution (230 mM TBAHS buffered with 130 mM sodium carbonate) and 10 mL 75% butyldiglyme isooctane extraction solvent. The mixture is shaken for 5 minutes and allowed to settle. The organic extract is separated out and then slurried with 1.0 gram of NaCl to dry the extract. The extract can then be reacted with sodium and quantified with FISE as described above. Results are provided in FIG. 8 and show an R2 of greater than 0.999 when regressed against the theoretical values.

### Example 3: SPE Direct Reaction

In this method, a solid phase extractant is reacted directly to remove fluorine atoms of adsorbed compounds without first transferring the analyte from the SPE to a solvent. Standards were made from AFFF concentrates in water at 10 ug/mL fluorine based off of direct spike fluorine measurements. These standards were used to directly spike a series of test samples in 10 mL volumes of deionized water at 0.5 to 10.0 ppm fluorine range in Dexsil test tubes. To this series 1.2 g conditioned SP70 polystyrene divinylbenzene solid phase SPE sorbent material was added, and the tubes were lightly vortexed for 20 minutes to reach equilibrium, creating a slurry. The SPC sorbent material particle size was between about 250.0 and 850.0 μm, with a pore volume of 1.1 mL/g, and a mean pore size of about 65 Å. The particles had a surface area of about 700.0 m²/g and a density of about 1.01 g/mL at 25 C (true wet). The sample/sorbent slurry was poured into a 10.0 mL polypropylene pipette barrel plugged with a fibrous polyester filter and the filtrate discarded. The filtered slurry was completely dried in air using a vacuum manifold. Dryness was determined in situ by monitoring the mass of select samples. Samples can be dried to less than 5%, less than 2%, less than 1% or less than 0.1% moisture by weight. After complete drying, the entire SPE sorbent material was poured into a reaction tube and 5.0 mL of a mixture of 20% (w/w) isooctane in BDG was added as solvent. A sodium reaction and quantification proceeded from this point using the above procedure.

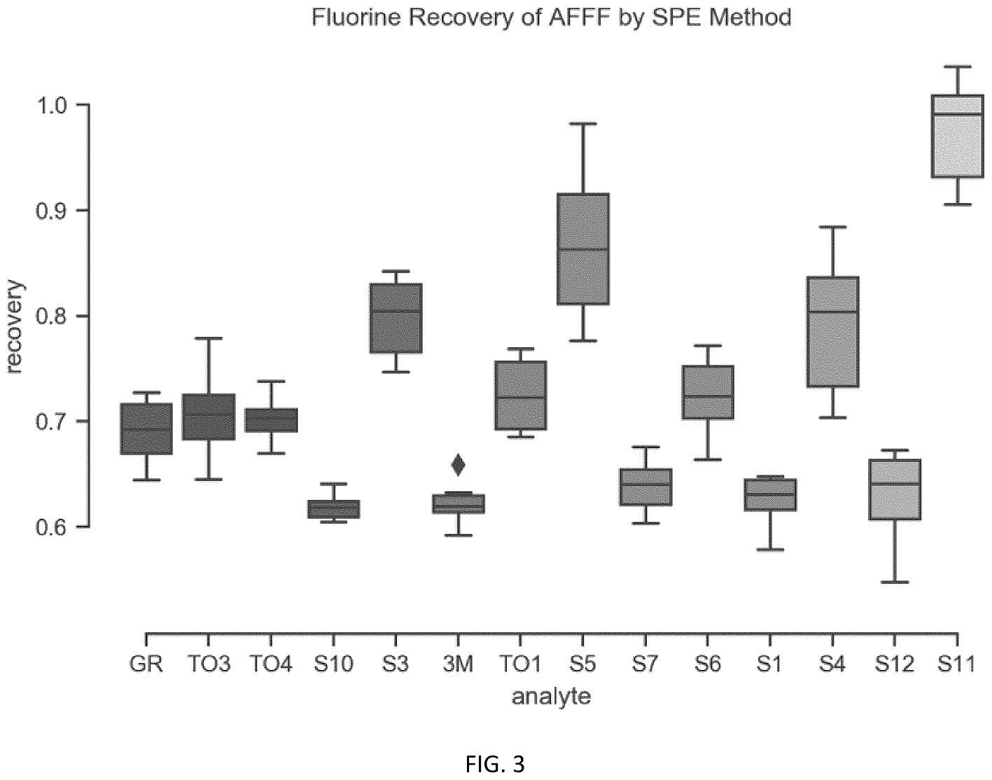

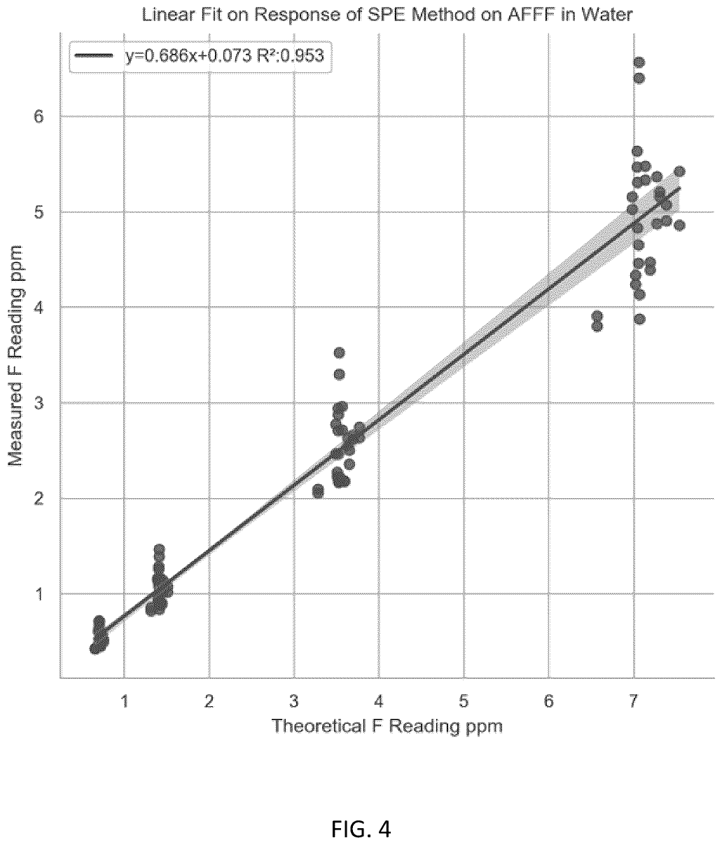

FIG. 3 is a graph showing recovery of AFFF spiked water by direct reaction SPE method with coded analyte when compared to direct injection fluorine content over 0.5-10 ppm total fluorine range. FIG. 4 is a plot and best fit line of the linear response of direct reaction SPE method compared to direct concentrate injection covering full real sample spike database.

The SPE extraction/direct reaction method performed well over all the AFFF foam concentrates and field samples tested when compared to the total fluorine load measured by direct spike experiment, as shown in FIG. 3, and demonstrated a correlated fluoride response across the required concentration range, as shown in FIG. 4.

It has been found that pH, AOT concentration and NaCl concentration are all important to achieve adequate recovery of fluorine. pH was also important for the anionic ion pairing. Some of the foam concentrates were difficult to analyze with less than 50% recovery of fluorine, and to target nonionic and polymeric PFAS along with ionic compounds a method was needed to extract PFAS compounds based on their universal surface activities. The disclosed SPE methods solve this issue. While SPE methods for the extraction of PFAS from water are known, elution into a solvent after loading the analyte sample on the SPE stationary phase has been a required step. The EPA PFAS water method 537 uses a column extraction method and 250 mL of sample to achieve very low detection levels. For the scope of measuring AFFF foam concentrate spills and remediation, only a ppb-ppm level was needed and therefore an extensive column procedure was unnecessary.

For the required detection levels, the samples can be shaken or vortexed with activated or conditioned hydrophobic SPE sorbent in order to adsorb the surface-active organic fluorine compounds to the sorbent. Other suitable mixing techniques include mechanical, pneumatic, jet, vibrational, magnetic, mixing devices with reversible movement of mixer parts, as well as static, rotary, rotor-stator, vortex, and ultrasonic devices. Conditioners for the SPE sorbent are preferably water miscible organic solvents, such as methanol or acetone. Additional or alternative solvents that can be used include glycerol; propylene glycol; ethanol; formic acid; acetic acid; methanol; 2-propanol; acetone; dimethyl sulfoxide; dimethylformamide; 1-propanol; ethylamine; acetonitrile; acetaldehyde; propanoic acid; furfuryl alcohol; methyl diethanolamine; ethylene glycol; 1,3-butanediol; butyric acid; tetrahydrofuran; 1,4-butanediol; dimethoxyethane; pyridine; 1,5-pentanediol; diethylenetriamine; diethanolamine; 2-butoxyethanol; triethylene glycol; 1,4-dioxane; 1,3-propanediol; 1,2-butanediol; methyl isocyanide; and n-methyl-2-pyrrolidone. The term hydrophobic includes materials having a water contact angle of larger than 90°. In various embodiments solvents can have a water contact angle of larger than 90°, larger than 120°, larger than 150°, and materials having a water contact angle between 90° and 180°.

After mixing the SPE sorbent with the sample, the excess amount of moisture retained by the solid phase was dried by pulling air over the SPE sorbent in a manifold design with a vacuum pump. After complete drying, the adsorbent was directly reacted in the reducing defluorination reaction described above. This direct reaction method was adopted because many AFFF formulations did not respond to attempts to elute them off of the SPE sorbent beads. It was determined that this was the best workup to measure the full breadth of AFFF concentrates spiked to water as the extraction process was nearly universal.

To analyze ground water for even smaller ppt levels, the SPE/direct reaction method described can be adapted to use a column collection method involving a larger sample size. Sample sizes of 100-500 mL would result in reducing the MDL by a factor of 10-50.

### Example 5: Low Level SPE Column Water Methods

This method uses a solid phase extraction step to concentrate the PFAS from an aqueous sample followed by a wash to displace any inorganic fluoride retained on the column. It is able to achieve an MDL of less than 1 ppb or less than 500 ppt, by weight. Following the concentration and wash, the PFAS are eluted with a polar solvent. To transfer any PFAS to a non-sodium reactive solvent, the elution solvent can be removed by drying and the residual sample can be dissolved or suspended in a non-sodium reactive solvent system. In one set of embodiments, the elution solvent can be placed in a reaction tube and the solvent removed by heating and/or passing a gas over the sample. Once the elution solvent has been removed, the non-sodium reactive solvent can be added. Once in the reaction solvent, the organic fluoride is liberated through a reductive defluorination reaction, such as a sodium reaction, extracted to buffer, and the fluoride concentration measured with a fluoride ion selective electrode (FISE) on Dexsil's L2000DXT unit. Due to the sample prep steps and the fact that it is a “total organic fluorine” method, this analysis produces a fluoride result that is “method defined” and is referred to herein as “adsorbable organic fluorine,” “extractable organic fluorine,” or both.

The strict attention to sampling techniques is an important part of the approved PFAS methods due to a number of factors including: The abundance PFAS background in everything from reagents and supplies to everyday items that may be present at a sampling site or in the lab, the low levels usually targeted, and the high surface activity of these compounds. For this study, all aqueous PFAS samples were prepared in polypropylene (PP) bottles previously shown to be fluoride free, using de-ionized (DI) water. Stock solutions were prepared using analytical grade Perfluorohexane sulfonate (PFH×S) purchased from Matrix Scientific at a concentration of 100 ppm fluorine wt/wt. Test samples were then prepared by diluting the stock solutions directly in 250 mL PP sample bottles. Solid phase extraction (SPE) columns were prepared either in glass or polypropylene support with either 40 mg granulated activated carbon (GAC) purchased from Metrohm (Cat #SNG-ICT0008 pore volume 1.2 g/mL, specific surface area 560 m2/g, pore radius 290 angstrom) or 500 μL macroporous polystyrene-divinylbenzene (PS-DVB) beads (Mitsubishi Chemical HP20SS). Prior to extraction samples are fortified with 0.5 mL 2M Potassium Nitrate. Samples are loaded to SPE columns with either vacuum manifold via HDPE reservoir or multichannel peristaltic pump with silicone microbore tubing. Both materials were previously demonstrated to be fluoride free. Loading is done at 3 mL per minute. After the full sample is loaded onto the column, all surfaces that had been in contact with the sample are washed with 25 mL 0.01M Potassium Nitrate (Sigma) to remove inorganic fluoride interferences. The wash solution is added to the sample vessel, shaken briefly, then pumped through the system following the sample loading path to the column. This is followed by a similar wash with DI water to remove the residual potassium nitrate. Samples are then eluted with 10 mL of 1% methanolic ammonium hydroxide, making sure to contact all surfaces used in sample preparation, and collected into the polyethylene reaction tube then dried under a gentle stream of nitrogen in warming manifold at 63° C. The dry samples are then ready for the defluorination reaction.

The reductive defluorination (RD) takes place in a fully contained vessel with premeasured quantities of reagents prepared in glass ampules. These ampules use catalyst, solvent, and metallic sodium to create an extremely reactive free radical sodium naphthenide that liberates fluorine from PFAS molecules. First, 5 mL of reaction solvent is added to the tube and capped. Then the catalyst ampule is broken and the mixture shaken for 10 seconds. Then, the sodium dispersion ampule is broken and the reaction is shaken intermittently for one minute. The reaction is quenched by adding 7 mL of buffer and shaking for 2 minutes. The aqueous extract is decanted to a fresh HDPE tube, allowed to equilibrate to room temperature, and read on the FISE-L2000DXT system.

A representative PFAS compound (PFH×S), was used to demonstrate the feasibility of the method. A stock solution of PFH×S was prepared at 100 μg/mL fluoride in DI water. The 250 mL water samples were prepared from stock solution and DI, at 0, 3, 6, 15.2, 30, and 60 ug/L fluorine. The method described above was used for SPEs of PS-DVB at a 500 uL column size, and for GAC at a 40 mg column size by peristaltic pump loading. A vacuum loading method was also used with PS-DVB and the same analyte.

To test how robust the method was to inorganic fluoride background, GAC columns were challenged with increasing levels of fluoride up to 16 mg/L in 250 mL DI water blanks fortified with potassium nitrate and given the same treatment as described in the method. Low level PFH×S samples were also prepared at 3.2 ug/L fluorine with and without an additional 8 mg/L inorganic fluoride. These samples were analyzed in triplicate using the standard method for both SPE materials.

PFH×S performed very well in the laboratory fortified blanks for both SPE materials and all loading methods. The linear range extended to the highest standard level tested at 60 ug/L for the 40 mg GAC columns. For the HP20SS PS-DVB material there was a roll-off in recovery at 60 ug/L, probably from column breakthrough but was very linear to the next lowest standard. All laboratory blank levels tested were below the MDL of the instrument even when challenged up to 16 mg/L in GAC. An increase in fluoride measurement was not measured in the low level PFH×S samples challenged with 8 mg/L inorganic fluoride. If anything, there was a slight depression in fluoride reading in the inorganic fluoride spiked samples, presumably from changing the conditions during the SPE loading, however, the readings were not statistically significant with P values well over 0.05 using a Student's T test. To estimate an MDL for the method, the readings from three samples in DI water blanks were combined with the three samples challenged with 8 mg/L, and used to calculate a conservative MDL for both SPEs respectively. The resulting MDLs (0.46 ug/L and 0.5 ug/L) are a good first estimate of the method sensitivity and are consistent MDLs established for the FISE with inorganic fluoride standards in an identical buffer.ⁱ

TABLE 1
Inorganic Challenge GAC Column

| Conc. | Conc. | F | Measured | | | |
|---|---|---|---|---|---|---|
| Inorg. | Org. | Reading | Conc. | | | |
| F mg/L | F ug/L | mg/L | ug/L | Recovery | Average | STDEV |
| 0 | 3.2 | 0.099 | 2.772 | 0.866 | 0.893 | 0.032 |
| 0 | 3.2 | 0.106 | 2.968 | 0.928 | | |
| 0 | 3.2 | 0.101 | 2.828 | 0.884 | | |
| 8 | 3.2 | 0.099 | 2.772 | 0.866 | 0.828 | 0.051 |
| 8 | 3.2 | 0.088 | 2.464 | 0.770 | | |
| 8 | 3.2 | 0.097 | 2.716 | 0.849 | | |
| | | ttest | 0.139 | | | |
| | | T* | 3.365 | | | |
| | | STDEV | 0.166 | | | |

| | |
|---|---|
| Estimated MDL | 0.558 |

TABLE 2
Inorganic Challenge PS-DVB Column

| | Concen- | | | | | |
|---|---|---|---|---|---|---|
| Conc. | tration | F | Measured | | | |
| Inorg. | Org. | Reading | Conc. | | | |
| F mg/L | F ug/L | mg/L | ug/L | Recove | Avera | STDE |
| 0 | 3.2 | 0.116 | 3.248 | 1.015 | 1.038 | 0.027 |
| 0 | 3.2 | 0.122 | 3.416 | 1.068 | | |
| 0 | 3.2 | 0.118 | 3.304 | 1.033 | | |
| 8 | 3.2 | 0.108 | 3.024 | 0.945 | 0.989 | 0.049 |
| 8 | 3.2 | 0.112 | 3.136 | 0.980 | | |
| 8 | 3.2 | 0.119 | 3.332 | 1.041 | | |
| | | ttest | 0.197 | | | |
| | | T* | 3.365 | | | |
| | | STDEV | 0.142 | | | |
| | Estimated | 0.478 | | | | |
| | MDL | | | | | |

### Example 6: Soil Extractions TBAHS Method

Recovering PFAS from soils has several issues, the first of which is that surface active analytes tend to be challenging to extract from high surface matrices such as soils. Secondly, soil contains inorganic fluoride well above the screening levels necessary for a useful FISE method. This requires a method to have a robust extraction procedure along with a separation mechanism to reject inorganic fluoride.

In order to maintain a robust extraction, anionic PFAS were targeted with the use of an ionic pairing solution combined with butyldiglyme solvent. The BDG solvent was amended with 20% isooctane (w/w) to minimize the moisture retained for the subsequent moisture sensitive reaction. The ionic pairing mechanism can be thought of as shielding the ionic head group with butyl groups, thus increasing the partition of anionic compounds into the nonpolar organic phase.

Soil types tested included sand, fresh local topsoil, and topsoil sampled in 1997 from under pine trees (pine topsoil). Pure PFAS analytes were dissolved in water at approx. 500 μg/mL fluorine using brief exposure to ultrasonic bath to aid dissolution. A volatile organic solvent was used to dissolve the 6:2 Fluorotelomer alcohol. In both cases a small molar excess of sodium carbonate was added to the mixture. These standard solutions were then used to directly spike 10 grams of soil in the range of 0.5-20 ug/g fluorine in 50 mL polypropylene centrifuge tubes. The moisture content was then normalized to 400 uL total with DI water to maintain constant moisture content across spike levels. The pine topsoil was completely dry so an additional 1.0 mL DI was added to maintain uniform moisture levels. Then, 3 mL of TBAHS (230 mM tetrabutylammonium hydrogen sulfate) buffered with 100 mM sodium carbonate were added, followed by 10 mL of a 20% (w/w) isooctane in butyldiglyme solution. The mixture was shaken for 5 minutes and allowed to separate. Finer soils were centrifuged. The organic layer was removed and slurried over a gram of dry sodium chloride. 5 mL of the organic layer was reacted with the sodium reagent as described above. The MDL was determined using 10 μL of PFAS standard made up at 500 μg/mL fluorine spiked into 10 g pine topsoil with eight replicates and eight blank replicates.

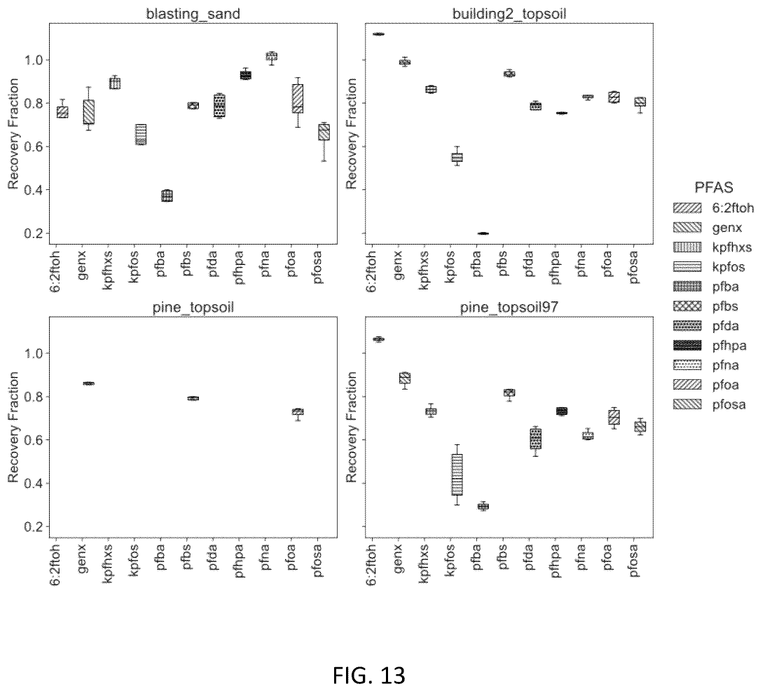

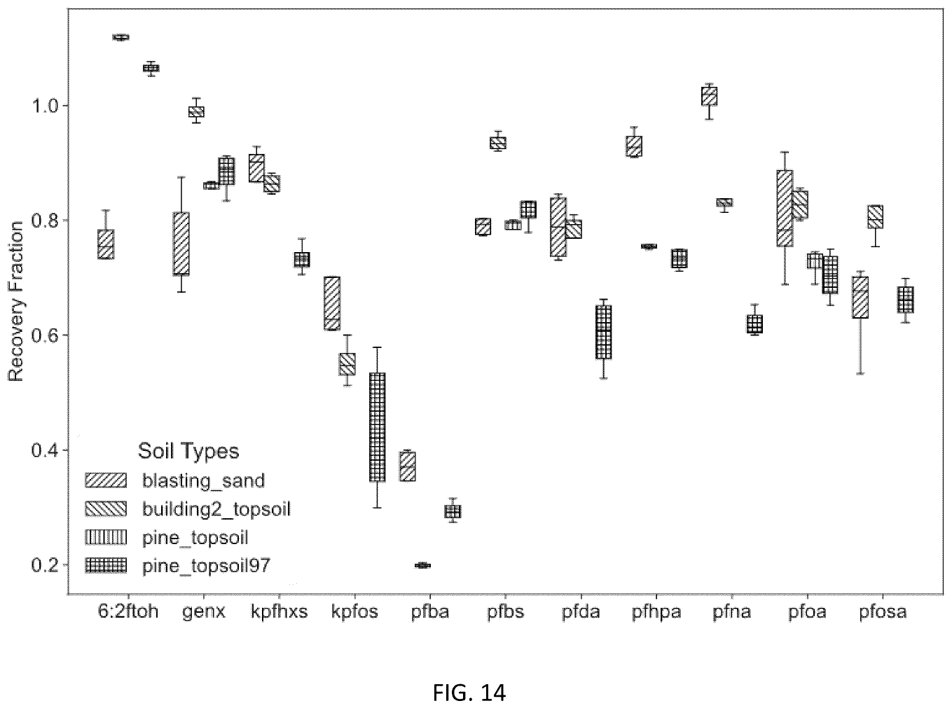

The regulated PFAS, cited above, demonstrate repeatable recoveries in four different soils over a range of concentrations. As can be seen in FIG. 13, there are two notable outliers to this trend; PFBA and PFOS. Plotting the same data, sorted by analyte in FIG. 14, shows clearly that both compounds were difficult to extract from all soil types. It is not clear why these two PFAS compounds were poor performers. It is not simply the head group, as other sulfonates did not present a problem, nor did the other carboxylic acids. One factor that may have played a role with the PFBA would be the fluorinated chain length. It is not clear how this plays into the extraction process, but PFBA has the lowest molecular weight of the analytes tested, is much more volatile, and may have lower partition potential. By contrast the C-4 sulfonate was one of the better performers and the C-8 carboxylic acid was the easer to extract analogue.

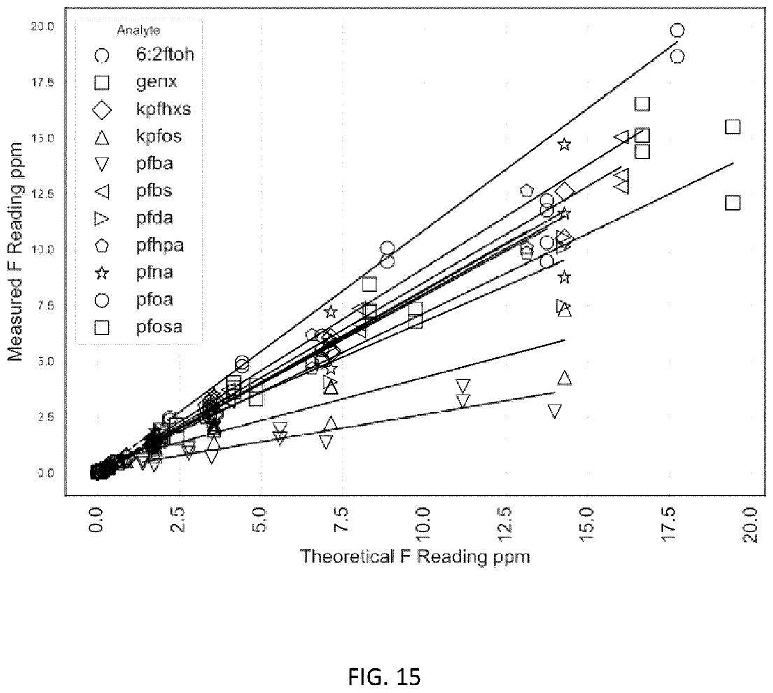

If we examine the data as a function of concentration for each analyte as in FIG. 15, it is clear that each of them has a different recovery. It can also be seen that for each analyte the recovery is linear-R2 values are all in the range of 0.9.

TABLE 3
PFAS Standards

| | Molecular | Fluorine |
|---|---|---|
| Analyte | Weight | Fraction |
| Perfluorooctanesulfonate (PFOS) | 538.22 | 0.60 |
| Perfluorodecanoic acid (PFDA) | 514.08 | 0.70 |
| 1,1,2,2,3,3,4,4,5,5,6,6,6- | 438.22 | 0.56 |
| tridecafluorohexane-1-sulfonate (PFHxs) | | |
| Perfluorononanoic acid (PFNA) | 464.08 | 0.70 |
| 1H,1H,2H,2H-perfluorooctanol | 364.1 | 0.68 |
| (6:2FTOH) | | |
| Perfluorooctansulfonamide (PFOSA) | 499.15 | 0.65 |
| Perfluorobutyric acid (PFBA) | 214.039 | 0.62 |
| Perfluorooctanoic acid (PFOA) | 414.07 | 0.69 |
| Nonafluorobutane-1-sulfonic acid (PFBS) | 300.1 | 0.57 |
| 2,3,3,3-tetrafluoro-2-(1,1,2,2,3,3,3 | 330.053 | 0.63 |
| heptafluoropropoxy)propanoic acid (GenX) | | |
| Perfluoroheptanoic acid (PFHpA) | 364.06 | 0.68 |

FIG. 13 shows sample recoveries for all analytes by soil type. FIG. 14 shows recoveries for all soil types by analyte. FIG. 15 shows all soil data plotted by individual analyte.

### Example 7: TBAHS Direct Ion Pairing Method with Methanol

In order to improve the long chain PFAS recovery from troublesome soils with the TBAHS extraction method a two-step extraction method was developed. This method uses an equal part mixture of methanol and the TBAHS ion pairing reagent to enhance the extraction of long chain anionic PFAS from Pine topsoil 1997. A modified drying method was developed since the methanol residues, which are not removed from NaCl slurry alone, would quench the reaction. It was found that a combination of increasing the alkane fraction of the organic extraction solvent to 50% isooctane in butyl diglyme and shaking the extract with a saturated solution of NaCl lowered the water and methanol level enough for a robust reaction.

The following experiment shows the increase in extraction efficiency with the addition of methanol for some of the longer chain PFAS. 10 g of soil was added to a 50 mL polypropylene centrifuge tube. 0.5 g of DI water was added and shaken for 5 minutes. 100 μL of aqueous PFAS standard was added and shaken for 5 minutes. 3 mL of methanol and 3 mL of 230 mM tetrabutylammonium hydrogen sulfate/100 mM sodium carbonate solution was added and shaken for 5 minutes. 12 mL of 1:1 v/v butyl diglyme:isooctane solution was added and shaken for 15 minutes. Tubes were briefly centrifuged and approximately 8 mL of soil free butyl diglyme:isooctane supernatant was transferred to a 20 mL tube. 2 g NaCl and 5 mL of DI water was added and shaken for 5 minutes then allowed to separate for 5 minutes. This step removed the methanol and water entrained in the butyl diglyme:isooctane phase. 5 mL of the butyl diglyme:isooctane was added to a reaction tube containing an ampule with 0.4 g of 20% Na metal suspension in light oil. 2 mL of 20 mg/mL dioctyl sulfosuccinate sodium salt (AOT) in butyl diglyme was added and shaken for 20 seconds. Sodium ampule was broken and tube was shaken intermittently for 2 minutes. Reaction was quenched with 7 mL of aqueous sodium acetate buffer and shaking for 2 minutes to extract fluoride. After venting the tube was inverted and allowed to stand on cap for 2 minutes which allowed organic reaction mixture and aqueous extract mixture phases to separate. Aqueous extract is passed through a fibrous, fluoride free filter into a new test tube and read with a calibrated FISE connected to an L2000 halogen detection system (Dexsil).

TABLE 4
% Average Recovery of PFAS in Soil with TBAHS or TBAHS + Methanol Method

| | PFBA | PFBS | PFHxS | PFHpA | PFOA | PFOS | PFNA | PFDA | GenX |
|---|---|---|---|---|---|---|---|---|---|
| TBAHS | 29% | 81% | 73% | 73% | 71% | 44% | 62% | 60% | 88% |
| TBAHS/ | 9.9% | 8.1% | 92.0% | 105.7% | 97.0% | 82.9% | 96.0% | 100.6% | 65.9% |
| MEOH | | | | | | | | | |

### Example 8: Two Step Acidic Method

One aspect of PFAS extraction from soil into an organic medium suitable for the defluorination reaction involves the transfer of the PFAS from the aqueous direct ion pairing solution to the organic defluorination reaction solvent occurring during the extraction step. Polarity of the PFAS affects this step so that the greater the polarity of the PFAS compounds the less they will partition into the organic reaction solvent and therefore lower recovery. This resulted in a lower recovery for the more polar short chain PFAS, perfluorobutyric acid and perfluorobutane sulfonic acid, with the TBAHS Direct Ion Pairing Method with Methanol. To correct this problem the aqueous portion of the soil extraction solution was acidified, which protonated the basic form of the PFAS and made them more non-polar. A good recovery from Ottawa sand was achieved for the PFAS compounds tested.

Extraction of PFAS from Ottawa sand using a lower pH extraction solution: 10 g of Ottawa sand was added to a 50 mL polypropylene centrifuge tube. Add 0.5 g of DI water and shake for 5 minutes. Add 100 μL of aqueous PFAS standard and shake for 5 minutes. Add 5 mL of 0.46 M sodium bisulfate solution adjusted to approximately pH 0.3 with sulfuric acid and shake 5 minutes. Add 12 mL of of 1:1 v/v butyl diglyme:isooctane solution and 2 g NaCl and shake for 15 minutes. Tubes were briefly centrifuged and 5 mL of soil free butyl diglyme:isooctane supernatant was transferred to a reaction tube containing an ampule with 0.4 g of 20% Na metal suspension in light oil. 2 mL of 20 mg/mL dioctyl sulfosuccinate sodium salt (AOT) in butyl diglyme was added and shaking 20 seconds. Sodium ampule was broken and tube was shaken intermittently for 2 minutes. Reaction was quenched with 7 mL of aqueous sodium acetate buffer and shaking for 2 minutes to extract fluoride. After venting the tube was inverted and allowed to stand on cap for 2 minutes which allowed organic reaction mixture and aqueous extract mixture phases to separate. Aqueous extract is passed through a fibrous, fluoride free filter into a new test tube and read with a calibrated FISE connected to an L2000 halogen detection system (Dexsil). The recoveries are presented in Table 5 below.

TABLE 5
% Recovery of PFAS in Soil with PFAS
Acidic Protonation Method

| PFBA | PFBS | PFOA | PFOS |
|---|---|---|---|
| 94% | 97% | 98% | 83% |

### Example 9: TOAB One Step Extraction

To remove any inorganic fluoride from the soil matrix, water is an integral part of the extraction process, however, due to the constraints of the defluorination reaction, to establish a robust reaction from a one step extraction, it is necessary to reject as much water and protic material as possible. This is achieved by two mechanisms: decreasing the polarity of the organic layer, demonstrated in the examples through increasing alkane fraction; and saturating the aqueous layer with salt. This procedure makes it feasible to extract a solid material such as soil and react the material without requiring an independent drying step.

As described above, an ionic pairing agent must be employed in order to obtain adequate recoveries for the anionic PFAS compounds. The TBAHS reagent used in the previous example provided only moderate recoveries for most of the PFAS compounds tested. Tetraoctylammonium bromide (TOAB), in contrast, provided excellent recovery at much lower concentrations. This method performed flawlessly over the breadth of PFAS tested in Ottawa sand which is indicative of how this method would perform unhindered by complex soil matrices. Soil sample Building 2 Topsoil also performed very well with this pairing agent. Both soils produced MDLs for PFHpA in soil below the fluoride electrode manufactures limit of detection indicating the Nernstian nonlinear deviation from the ideal system as the limiting factor. Pinetop 97 was a soil in which short chains performed well while compounds such as PFOS and the longer chain PFAS were recovered poorly. This poor recovery was not an issue with equilibrium but through a kinetic mechanism as demonstrated through a 24 hour extraction experiment that ameliorated the low recoveries. Blank samples for all soils tested consistently low and when the method was stressed by spiking blanks with 1000 μg of inorganic fluoride to Ottawa sand 99.93% of the inorganic fluoride was rejected.

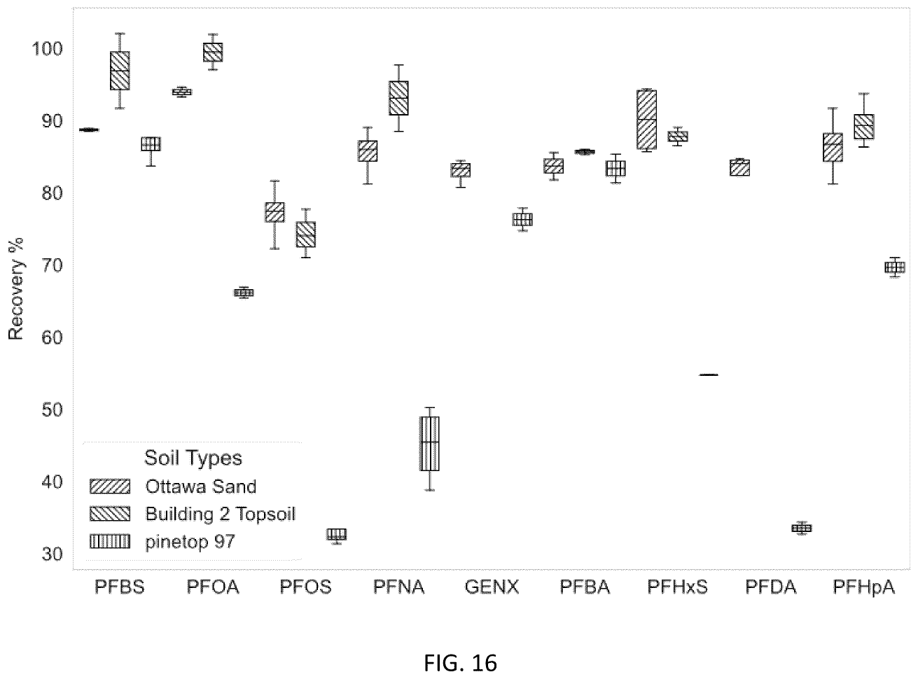

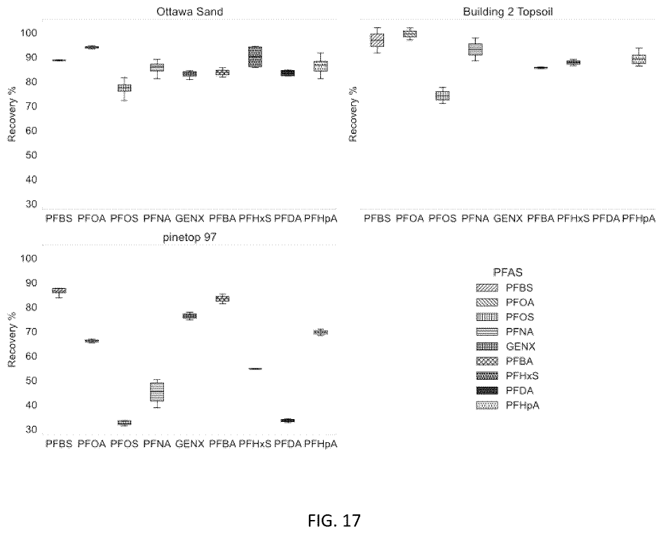

To each of several 50 mL polypropylene centrifuge tubes 10 grams of soil were added and then for the purposes of sensitivity, precision, and recovery determination 0-100 uL of a 100 μg/mL aqueous PFAS standards were added. Once samples were produced in this way the extraction reagents were added all at once. This consisted of 2.9 mL deionized water, 5 mL isooctane, 5 mL BDG, 2 g NaCl, and 100 uL 38% TOAB in MeOH. The centrifuge tubes were capped and shaken for 5 minutes to extract the analytes. The mixture was then briefly centrifuged to establish clear separation between the aqueous and organic phases. Once separated, 5 mL of the organic phase was added to a reaction tube and sealed. The naphthalene and AOT (sodium docusate) in BDG catalyst ampule is broken, shaken for 10 seconds, followed by breaking the sodium ampule and shaken for 1 minute intermittently. At the one minute mark the reaction tube is opened and 7 mL of buffer solution added, recapped, and shaken for 2 minutes to quench the reaction and extract the liberated fluoride. After 2 minutes the tube is vented, flipped on its cap, and allowed to separate for 5 minutes. The aqueous layer is then let out through the turret cap, passed through a filter into a new tube for fluoride measurement. FIG. 16 shows the recovery for all analytes across all soil types tested. FIG. 17 illustrates the recovery for various PFAS compounds in the three different types of soil.

TABLE 6
MDL determination for TOAB one step extraction
method of Ottawa Sand for PFHpA.

| Ottawa Sand | F-Reading ug/mL | F Conc. ug/g | Recovery % |
|---|---|---|---|
| | 0.127 | 0.173 | 86.3 |
| | 0.129 | 0.176 | 87.8 |
| | 0.122 | 0.165 | 82.7 |
| | 0.128 | 0.174 | 87.1 |
| | 0.120 | 0.162 | 81.2 |
| | 0.125 | 0.170 | 84.9 |
| | 0.126 | 0.171 | 85.6 |
| | 0.112 | 0.151 | 75.3 |
| Average | 0.124 | 0.168 | 83.9 |
| Stdev · s | 0.006 | 0.008 | |
| t* | 2.998 | 2.998 | |
| MDL | 0.017 | 0.025 | |

TABLE 7
MDL determination for TOAB one step extraction method
of Building 2 Topsoil for PFHpA.

| Building | | | |
|---|---|---|---|
| 2 Topsoil | F-Reading ug/mL | F Conc. ug/g | Recovery % |
| | 0.127 | 0.173 | 86.3 |
| | 0.130 | 0.177 | 88.6 |
| | 0.131 | 0.179 | 89.3 |
| | 0.128 | 0.174 | 87.1 |
| | 0.131 | 0.179 | 89.3 |
| | 0.137 | 0.187 | 93.7 |
| | 0.131 | 0.179 | 89.3 |
| | 0.136 | 0.186 | 93.0 |
| Average | 0.131 | 0.179 | 89.6 |
| stdev · s | 0.004 | 0.005 | |
| t* | 2.998 | 2.998 | |
| MDL | 0.011 | 0.015 | |

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items, while only the terms “consisting of” and “consisting only of” are to be construed in the limitative sense.

1-51. (canceled)

52. A method for detecting per and polyfluoroalkyl substances (PFAS) in a soil sample, the method comprising:
mixing the soil sample with an aqueous solution to produce a mixture;
adding a solvent comprising a non-sodium reactive solvent to the aqueous solution or to the mixture;
agitating the mixture;
reacting an organic layer from the mixture with an alkali metal organic compound; and
measuring a concentration of fluoride present in the organic layer.

53. The method of claim 52 wherein the aqueous solution comprises an alcohol.

54. The method of claim 53 wherein the aqueous solution has a pH of less than 3.

55. The method of claim 53 wherein the aqueous solution has a pH of greater than 10.

56. The method of claim 52 wherein the aqueous solution comprises a salt at a concentration of at least 1 M.

57. The method of claim 52 wherein the aqueous solution comprises a quaternary ammonium salt.

58. The method of claim 57 wherein the quaternary ammonium salt comprises tetrabutylammonium hydrogen sulfate and/or tetraoctylammonium bromide.

59. The method of claim 56 wherein the aqueous solution comprises a monovalent alkali metal salt.

60. The method of claim 52 wherein the aqueous solution is provided in an amount from 10% to 50% of the mass of the soil.

61. The method of claim 52 wherein the aqueous solution further comprises at least one of a surfactant, a salt, an acid, a base and an alcohol.

62. The method of claim 52 wherein the non-sodium reactive solvent comprises an aprotic polar solvent.

63. The method of claim 62 wherein the non-sodium reactive solvent comprises an aliphatic, cycloaliphatic, or aromatic hydrocarbon.

64. The method of claim 52 wherein the non-sodium reactive solvent comprises a mixture of an aprotic polar solvent and a hydrocarbon, the hydrocarbon to aprotic polar solvent ratio being between 1:10 and 1:2 on a wt/wt basis.

65. The method of claim 52 wherein the aqueous solution and non-sodium reactive solvent are comingled and are mixed with the soil sample concurrently.

66. The method of claim 65 wherein the aqueous solution comprises a salt at a concentration of at least 1 M and the non-sodium reactive solvent comprises a glycol diether and a hydrocarbon.

67-75. (canceled)
